SEQUENCE LISTING (9 sequences)

SEQ ID NO: 1 - protein, 115 aa, Artificial Sequence
  Synthetic
ASRDVVMTQSPLSLPVTPGEPASISCRSSQSIVHSNGNTYLEWYLQKPGQSPQLLIYKASNRFSGVPDRFSGSGSGTDFTLKISRVEAEDVGVYYCFQGSRLGPSFGQGTKLEIK

SEQ ID NO: 2 - protein, 117 aa, Artificial Sequence
  Synthetic
EVQLVESGGGLVQPGGSLRLSCAASGFTFSSFGMHWVRQAPGKGLEWVAYISRGSSTIYYADTVKGRFTISRDNAKNSLYLQMNSLRAEDTAVYYCARGITTALDYWGQGTLVTVSS

SEQ ID NO: 3 - protein, 326 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 4 - DNA, 25 bp, Artificial Sequence
  Synthetic
tatggcttctagagatgtggtgatg

SEQ ID NO: 5 - DNA, 82 bp, Artificial Sequence
  Synthetic
tgcagccaccgtacgcttgatctccagcttggtgccctggccaaaggtggggggcacmnnmnnmnnmnnmnngcagtagtag

SEQ ID NO: 6 - DNA, 70 bp, Artificial Sequence
  Synthetic
tgcagccaccgtacgcttgatctccagcttggtgccctggccaaamnnmnnmnnmnnmnngctgccctgg

SEQ ID NO: 7 - DNA, 24 bp, Artificial Sequence
  Synthetic
aggcggccctcgaggaggtgcagc

SEQ ID NO: 8 - DNA, 83 bp, Artificial Sequence
  Synthetic
agaccgatgggcccttggtggaggcgctggacacggtcaccagggtgccctggccccamnnmnnmnnmnnmnnggtgatgccc

SEQ ID NO: 9 - DNA, 92 bp, Artificial Sequence
  Synthetic
agaccgatgggcccttggtggaggcgctggacacggtcaccagggtgccctggccccagtagtccagmnnmnnmnnmnnmnnccgggcacag

(19) United States
(12) Patent Application Publication
(10) Pub. No.: US 2013/0052670 A1
(43) Pub. Date: Feb. 28, 2013

(54) METHOD FOR DETECTION OF AMYLOID BETA OLIGOMERS IN A FLUID SAMPLE AND USES THEREOF

(71) Applicants: Mary Savage, West Point, PA (US); Paul Shughrue, West Chester, PA (US); Abigail Wolfe, Ambler, PA (US); Alexander McCampbell, Chalfont, PA (US)

(72) Inventors: Mary Savage, West Point, PA (US); Paul Shughrue, West Chester, PA (US); Abigail Wolfe, Ambler, PA (US); Alexander McCampbell, Chalfont, PA (US)

(73) Assignee: MERCK, Rahway, NJ (US)

(21) Appl. No.: 13/544,554

(22) Filed: Jul. 9, 2012

Related U.S. Application Data
(60) Provisional application No. 61/507,332, filed on Jul. 13, 2011.

(51) Int. Cl. G01N 33/566; G01N 21/64

(52) U.S. Cl. 435/7.94; 436/501

(57) ABSTRACT

The invention herein is directed to a selective Aβ oligomer immunoassay capable of reliably and sensitively detecting Aβ oligomers in a biological sample of a patient. In one embodiment the inventive assay uses a pair of anti-AP oligomer antibodies, 19.3 and 82E1, to detect and quantify Aβ oligomers in a cerebrospinal fluid (CSF) sample. The inventive assay can be used to differentiate Alzheimer's disease (AD) patients from non-AD patients and/or to stratify AD patients according to the severity of their disease. The inventive assay can also be used as a target engagement assay that can measure bound Aβ oligomers as a surrogate end-point for the assessment of therapeutic efficacy and/or target engagement.

### FIELD OF THE INVENTION

The present invention relates to a method for the detection of amyloid beta (Aβ) oligomers associated with Alzheimer's disease (AD) in a biological sample. The invention also provides methods for diagnosing and evaluating treatments for AD.

### BACKGROUND OF THE INVENTION

Alzheimer's disease (AD) is a devastating neurodegenerative disease characterized by amyloid β (Aβ) plaque accumulation in brain regions involved in learning and memory. While these large insoluble plaques were once thought to cause AD, evidence now indicates that small diffusible oligomers of Aβ may be responsible. Amyloid-derived diffusible ligands (ADDLs) are a species of Aβ oligomers that can be generated in vitro with properties similar to endogenous Aβ oligomers (U.S. Pat. No. 6,218,506; Klein, et al., 2004, Neurobiol. Aging 25:569-580; Lambert, et al., 1998; Proc. Natl. Acad. Sci. U.S.A., 95:6448-6453. Aβ oligomers are present in the brain of AD patients, they bind neurons, and they induce deficits in neuronal morphology and memory. Studies with antibodies that bind Aβ oligomers have shown improvement in both neuronal morphology and memory.

While assays to measure Aβ monomers are known, which use the activity of β- and γ-secretase enzymes on the amyloid precursor protein (APP), few assays have been reported that specifically and reliably detect AP oligomers in a human fluid sample, such as cerebrospinal fluid (CSF), in both normal control and in AD (Georganopoulou, et al., 2005, Proc. Natl. Acad. Sci. U.S.A., 102:2273-2276; Fukumoto, et al., 2010, FASEB J., 24:2716-2726; Gao, et al., 2010, PLoS One, 2010 Dec. 30; 5 (12):e15725). Reported Aβ oligomer assays have employed a number of approaches, including ADDL-specific antibodies coupled with a bio-barcode PCR amplification platform (Georganopoulou, et al., 2005), overlapping epitope ELISAs (Gandy, et al, 2010, Ann. Neurol., 68:220-230; Xia, et al., 2009, Arch. Neurol., 66:190-199), also paired first with size exclusion chromatography (Fukomoto, et al., 2010), and amyloid-affinity matrices methods (Gao, et al., 2010; Tanghe, et al., 2010, Int. J. Alz. Dis., Sep. 2, pii: 417314), followed by oligomer dissociation and measurement with antibodies to Aβ monomers.

Aβ oligomers have also been detected using gel electrophoresis followed by western blot from either CSF or brain (Klyubin et al., 2008, J. Neurosci., 28:4231-4237; Hillen, et al., 2010, J. Neurosci., 30:10369-10379), or subsequent to size exclusion chromatography (Shankar, et al., 2011, Methods Mol. Biol., 670:33-44), relying on the molecular weight of oligomers that are maintained after the electrophoretic procedure. However, electrophoretic and blotting techniques do not provide the sensitivity required to see these species in normal control CSF (Klyubin, et al., 2008). Further, the findings by Georganopoulou demonstrate a 1000-fold range of Aβ oligomer concentrations and represent the concentration as fM. Aβ oligomer species represent a wide range of molecular weights and, as such, assignment of a precise molarity is problematic. The Georganopoulou assay is semi-quantitative and exhibits an analytical target concentration range of three orders of magnitude, with a lower limit of detection at 100 aM. Most reported methods (Georganopoulou, et al. 2005; Gao, et al., 2010; Fukumoto, et al., 2010; Gandy, et al., 2010) did not assess selectivity between signals from Aβ oligomers as compared to Aβ monomers, so the concentrations noted need to be viewed with caution. The Xia assay (Xia, et al., 2009, Arch. Neurol., 66:190-199), assay as marketed by Immunobiological Laboratories, Inc. (Minneapolis, Minn.) claims 320 fold selectivity for their Aβ1-16 dimers as compared to Aβ40 monomer, but lacks the selectivity needed to avoid cross-reactivity with Aβ monomer in the CSF. As Aβ oligomers in the CSF are hypothesized to be present at fM levels and CSF Aβ monomers are present between 1.5-2 nM, an assay that selectively measures Aβ oligomers in a CSF sample must have exceptional selectivity for Aβ oligomers over monomers.

In addition to measuring Aβ oligomer levels within human CSF as a potential disease biomarker, Aβ oligomers have also been used as a target for therapeutic monoclonal antibodies to treat AD (see, for example, U.S. Pat. Nos. 7,811,563, 7,780,963, and 7,731,962). It is believed that these antibodies access the CNS and clear the toxic ADDL species from the brain, through 1) catalytic turnover by Fc-mediated activation of microglia, 2) clearance of antibody/ADDL complexes into the cerebro-vasculature, or 3) enzymatic digestion of the ADDLs following antibody binding and improved access of degradative enzymes, such as neprilysin, insulin-degrading enzyme, plasmin; endothelin-converting enzymes (ECE-1 and -2), matrix metalloproteinases (MMP-2, -3 and -9), and angiotensin-converting enzyme (ACE). Thus, a goal of a selective Aβ oligomer assay is to measure the pharmacodynamic (PD) change in central nervous system Aβ oligomers following treatment with an anti-oligomer antibody or other treatment that alters Aβ monomer/oligomer formation or clearance. Additionally, an assay that would specifically enable the detection of Aβ oligomers bound to an anti-Aβ oligomer antibody, i.e., a target engagement (TE) assay, would be invaluable for the assessment of the therapeutic antibody following treatment.

The present invention provides for such assays that are capable of reliably and sensitively detecting Aβ oligomers in a human fluid sample.

### SUMMARY OF THE INVENTION

The present invention is directed to a selective Aβ oligomer assay capable of reliably and sensitively detecting Aβ oligomers in a biological sample, i.e. fluid sample, of a patient. The inventive assays use a pair of highly selective anti-Aβ oligomer antibodies, 19.3 and 82E1, to detect and quantify Aβ oligomers in a cerebrospinal fluid (CSF) sample. In one embodiment, the invention is a selective Aβ oligomer pharmacodynamic (PD) assay that can differentiate Alzheimer's disease (AD) patients from non-AD patients and/or stratify AD patients according to the severity of their disease. In yet another embodiment, the invention is a selective Aβ oligomer target engagement (TE) assay that can measure bound Aβ oligomers as a surrogate end-point for the assessment of therapeutic efficacy.

### BRIEF DESCRIPTION OF THE DRAWINGS

FIGS. 1A-1C are graphic representations showing the selectivity of the anti-ADDL antibody, 19.3, binding to the ADDL species of Aβ oligomers (middle bar of each set), as compared to Aβ monomer or Aβ fibril. FIG. 1A shows the ELISA binding of a panel of humanized (h3B3) and affinity matured anti-ADDL (14.2, 7.2, 11.4, 9.2, 13.1, 17.1, and 19.3) antibodies and three comparator antibodies (Comp 1, 2, and 3) to monomeric Aβ, ADDLs and fibrillar Aβ. Comparative antibody 2 is known to be non-selective antibody for ADDLs. The background of this assay was determined by removing the capture antibody from the ELISA (no mAb). Error bars represent standard error of the mean. FIG. 1B shows, in a one-sided ELISA with plates coated with either Aβ oligomer (▴) or Aβ monomer (), the relative affinities and maximum binding characteristics of the humanized antibody 19.3. FIG. 1C shows a competitive ELISA and the relative affinities of 19.3 for Aβ oligomers (▴) and Aβ monomer () coated on an ELISA plate in the presence of the competing species in solution.

FIGS. 2A-2C are graphic representations showing the sensitivity of three pairs of antibodies in a sandwich ELISA format using chemiluminesence (EnVision® Multilable Reader, Perkin Elmer, Waltham, Mass.), as the detection method and their relative affinities for Aβ oligomers. FIG. 2A shows depicts the anti-Aβ oligomer antibody 19.3 as the capture antibody and 82E1 as the detection antibody over a range of Aβ oligomer concentrations. FIG. 2B and 2C depict 6E10 and 19.3, respectively, as both the capture and detection antibodies. The 19.3×82E1 sandwich ELISA pair (FIG. 2A) was significantly more sensitive in detecting Aβ oligomers as compared to other pairs (FIGS. 2B and 2C).

FIG. 3 is a graphic representation of the sensitivity and selectivity for the detection of Aβ oligomers () as compared to Aβ monomer (▴) using the anti-Aβ oligomer antibodies 19.3 and 82E1 as measured using a paramagnetic micro-particle detector, such as the Erenna® digital detector (Singulex®, Almeda, Calif.). Use of the paramagnetic micro-particle detector significantly improved the sensitivity to detect Aβ oligomers with the 19.3/82E1 antibody pair.

FIGS. 4A and 4B are graphic representations of the levels of Aβ oligomers detected in human cerebrospinal fluid (CSF) samples. FIG. 4A shows that the Aβ oligomers levels were four fold higher in AD patients as compared to age matched control, i.e., non-AD, patients in a blinded evaluation using the inventive method herein. The differences were statistically significant to p≦0.0004 as determined using a two-way t-test and Mann Whitney analysis of ranks, assuming the population was non-Gaussian. FIG. 4B shows that the Aβ oligomer levels were eight fold higher in AD patients as compared to young control, i.e., non-AD, patients in a blinded evaluation using the inventive method herein. The differences were also statistically significant between these groups using the same statistical method as in FIG. 4A to a p-value≦0.0021.

FIGS. 5A and 5B are graphic representations of Aβ monomer levels in the CSF of either clinically confirmed AD or young control, i.e. non-AD, patients, with a corresponding decrease in the levels of Aβ42 monomer and unchanged levels of Aβ40 monomer in the AD samples. This is representative of the general pattern observed for AD patients and confirmed the disease state of the samples evaluated in FIG. 4B. FIG. 5A shows the reduced levels of Aβ42 monomer in the AD CSF samples. The differences were statistically significant to p≦0.002 as determined using a two-way t-test and Mann Whitney analysis of ranks, assuming the population was non-Gaussian. FIG. 5B shows the unchanged levels between the two groups of Aβ40 monomer.

FIG. 6 is a graphic representation of the correlation between Mini-Mental State Exam (MMSE) scores, as a measure of cognitive performance, and levels of Aβ oligomer measured using the inventive assay described herein. All patients depicted in FIG. 4B were included in this correlation. The correlation at −0.7445 pg/mL of Aβ oligomers was significant with p≦0.0001.

FIGS. 7A and 7B are graphical representations of the target engagement assay. FIG. 7A is a representation of anti-Aβ oligomer antibody 19.3/Aβ oligomer complexes formed ex vivo with spiking into human CSF () or Casein buffer (▴). FIG. 7B is a representation of anti-Aβ oligomer antibody 19.3/Aβ oligomer complexes formed ex vivo with spiking into human CSF () or Casein buffer (▴). Differential sensitivity was observed in the detection of 19.3/Aβ oligomer complexes in an anti-human kappa chain (capture)×82E1 (detection) target engagement ELISA (Example 9). The anti-kappa capture antibody poorly differentiated the anti-Aβ oligomer antibody 19.3 from the endogenous antibody species in human CSF.

FIG. 8 is a graphical representation of the PK of anti-ADDL antibody 19.3 assessed in primate (three male rhesus monkeys) cerebrospinal fluid (CSF) using a cisterna magna ported rhesus model following administration of a bolus IV dose of 20 mg/kg. At about 24 hours post dose, antibody 19.3 was present in the CSF at 100 ng/mL.

FIGS. 9A and 9B are graphic representations of the Aβ oligomer sandwich ELISA, i.e. the Pharmacodynamic (PD) Assay, and the Aβ oligomer/antibody sandwich ELISA, i.e. the Target Engagement Assay, respectively.

### DETAILED DESCRIPTION OF THE INVENTION

Applicants herein provide methods capable of reliably and sensitively detecting Aβ oligomers in the CSF of a patient for use as both a pharmacodynamic and target engagement measure of Aβ oligomers. The inventive methods can differentiate AD from non-AD patients and stratify AD disease state based on elevated levels of CNS Aβ oligomers in the AD patients, similar to uses previously reported for a tau/Abeta42 CSF ratio (De Meyer, et al., 2010, Arch. Neurol., 67:949-56). Moreover, an Aβ oligomer assay, detecting the most neurotoxic species, may correlate better and be a more dynamic measure of changes in cognitive performance, as compared to the poor correlation observed for levels of Aβ monomer. Applicants demonstrate herein for the first time that a peripherally administered anti-Aβ oligomer antibody can penetrate the blood-brain-barrier and bind Aβ oligomers and, when used in the inventive methods herein, can provide a surrogate end-point assay for the assessment of AD therapeutics.

Applicants herein have developed a highly sensitive assay to detect and measure the levels of a neuronally derived protein in a biological sample, i.e., a fluid sample, and uses thereof. In one embodiment of the invention, the neuronally derived protein is an Aβ oligomer and the fluid sample is a cerebrospinal fluid (CSF) sample. The inventive method uses two selective anti-Aβ oligomer antibodies in a sandwich ELISA using paramagnetic micro-particle detection. While Aβ oligomers have been found in biological samples, particularly in CSF (Georganopoulou, et al., 2005; Klyubin, et al., 2008), the limits associated with known detection methods (including both sensitivity and selectivity) have not enabled the reliable detection, let alone, quantification of Aβ oligomers for use to classify the disease state of the patient or for the development of AD therapeutics. Using two anti-Aβ oligomer antibodies,19.3 and 82E1, along with paramagnetic micro-particle detection, Applicants herein were able to develop a sandwich ELISA assay to detect Aβ oligomers in a biological sample to a limit of detection of 40 fg/mL. Using this assay, Applicants herein demonstrate highly significant elevations in Aβ oligomers in clinically confirmed AD samples as compared to either young or age-matched controls. These same samples were used to measure levels of Aβ42 and Aβ40 monomer and confirmed that in the AD samples Aβ42 monomer was significantly reduced as compared to the controls, while the Aβ40 monomer levels were unchanged. The inventive Aβ oligomer sandwich ELISA assay demonstrated significant correlations between Aβ oligomer concentration and performance on a cognitive test widely used to measure AD severity, known as the Mini-Mental State Exam (MMSE); the higher the cognitive score (up to a value of 30, which is cognitively normal) the lower the level of Aβ oligomer in the CSF. The inventive Aβ oligomer sandwich ELISA assay can be utilized with additional patient samples to generate further correlations with known fluid, imaging and cognitive biomarkers.

In addition to the pharmacodynamic assay above, Applicants have developed a target engagement (TE) having selectivity for a human IgG2/anti-Aβ oligomer complex such that it can be used with human CSF samples. As described in the examples that follow, the TE assay described herein overcomes the challenge of selectively distinguishing a non-native human IgG2 antibody (an anti-Aβ oligomer, IgG2 antibody) from the plethora of endogenous IgG antibodies present in human CSF. The selectivity of the TE assay was achieved by using a highly selective anti-IgG2-isotype capture (Southern Biotech, Birmingham, Ala., #9060-05), an antibody capable of capturing an Aβ oligomer IgG 2 antibody/Aβ oligomer complex from among the endogenous IgG2 species present in human CSF. The detection of Aβ oligomer bound to the 19.3/IgG2 isotype antibody was accomplished with a commercial antibody, 82E1 (Immunobiological Laboratories, Inc., Minneapolis, Minn.). This approach enabled reliable and consistent detection of the 19.3-IgG2 antibody/Aβ oligomer complexes, whether in buffer, in extracts of transgenic Tg2576 brain from animals treated with an Aβ oligomer antibody, or in human CSF samples spiked with an exogenous antibody and Aβ oligomer.

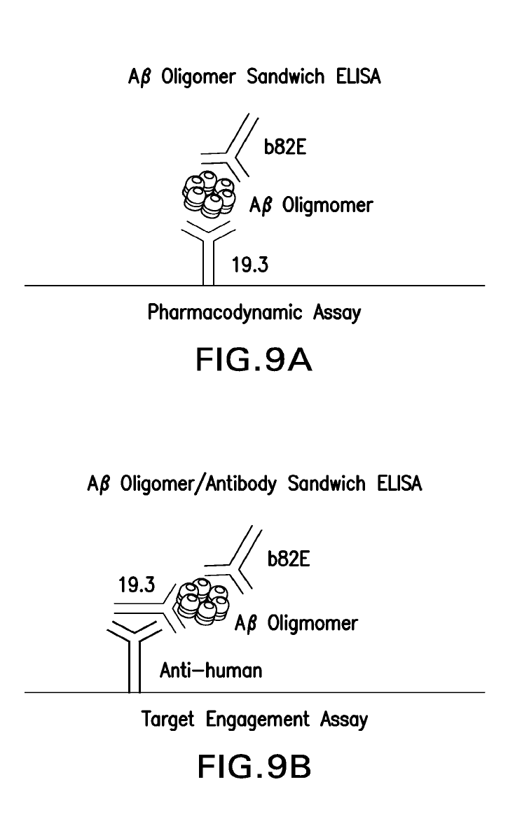

To enable an assay of unique sensitivity to detect the complexes of a therapeutic anti-Aβ oligomer IgG2 antibody bound to an Aβ oligomer, the anti-human IgG2 antibody is bound to a magnetic microparticle (MP) as described in the pharmacodynamic (PD) assay below. The MP/anti-human IgG2 complex is mixed with a CSF sample taken from an individual that was dosed with a therapeutic anti-Aβ oligomer antibody of the IgG2 isotype (therapeutic IgG2 antibody). This therapeutic anti-Aβ oligomer antibody will be bound to any Aβ oligomer species present in the CSF sample of the individual. This MP/anti-IgG2/anti-Aβ oligomer/Aβ oligomer complex is mixed with a second anti-Aβ oligomer antibody, 82E1, to which a fluorescent dye (fluor) is attached. The MP/anti-IgG2/anti-Aβ oligomer/Aβ oligomer/82E1-fluor complex is washed well by virtue of the magnetic properties of the microparticles and the 82E1-fluor complex is separated from the beads to reduce background. Single molecules of the 82E1-fluor represent the original levels of anti-Aβ oligomers/Aβ oligomer complexes that were present in the CSF of the dosed individual. This assay would enable confirmation that the therapeutic IgG2 antibody was engaging the Aβ oligomer target (FIG. 9B). With clearance of the Aβ oligomers during treatment, the therapeutic IgG2 antibody would engage fewer Aβ oligomers and thereby exhibit a reduced signal. Thus, the target engagement assay would enable a measure of efficacy for the therapeutic antibody being evaluated. The pharmacodynamic assay (FIG. 9A) would also exhibit a reduced signal, which would be attributed to the reduced presence of Aβ oligomers, such as after treatment. Accordingly, the pharmacodynamic assay can be used as an end-point surrogate for the evaluation of the efficacy of any therapeutic used for the treatment of AD.

The invention herein is a sensitive and selective sandwich ELISA assay which detects and quantifies endogenous Aβ oligomers in CSF samples from both AD and human control individuals. Development of the inventive assay began with the identification of a mouse hybridoma producing antibodies selective for Aβ oligomers over both Aβ monomers and fibrils. The selective anti-Aβ oligomer antibody, developed by Applicants (co-pending application PCT/US2011/______, claiming priority to U.S. Ser. No. 61/364,210) and referred to herein as 19.3, was humanized to an IgG2 isotype and was further characterized for affinity to Aβ oligomers by a one-sided ELISA, with an EC50 of approximately 1.6 nM. Further evaluations of the affinity of the 19.3 antibody for ADDLs in solution and in solid phase, as compared to Aβ monomer, demonstrated that 19.3 had approximately 600 times greater selectivity for Aβ oligomers than when evaluated in a competitive ELISA format. The sensitivity and selectivity of 19.3 for Aβ oligomers suggested a potential utility in a sandwich ELISA for Aβ oligomer detection.

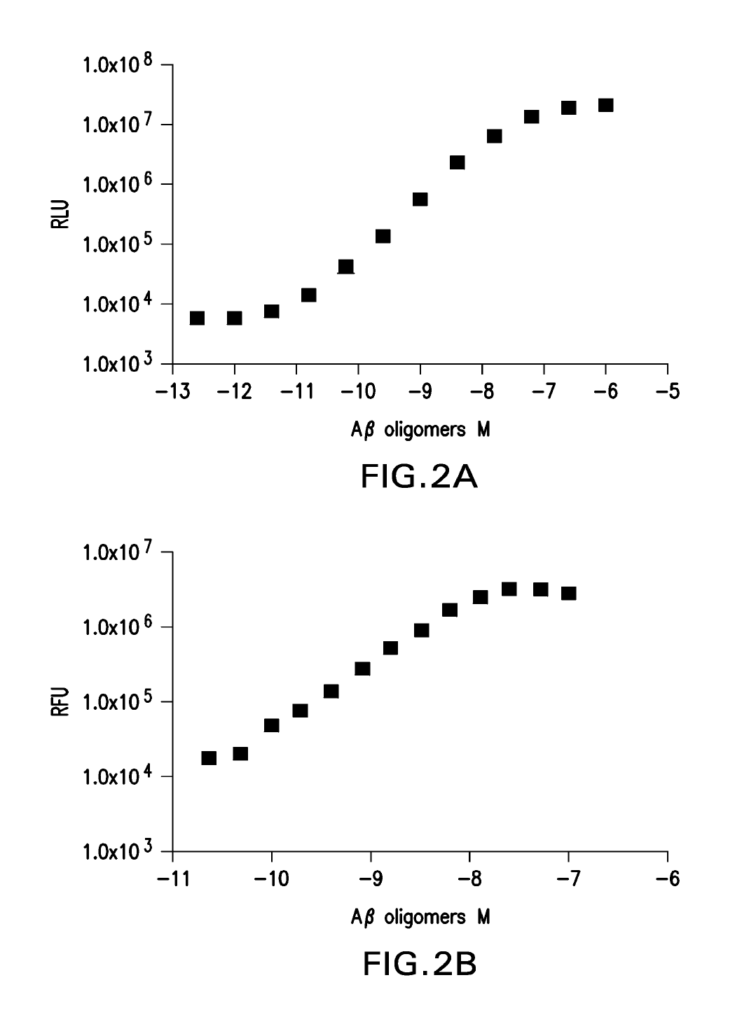

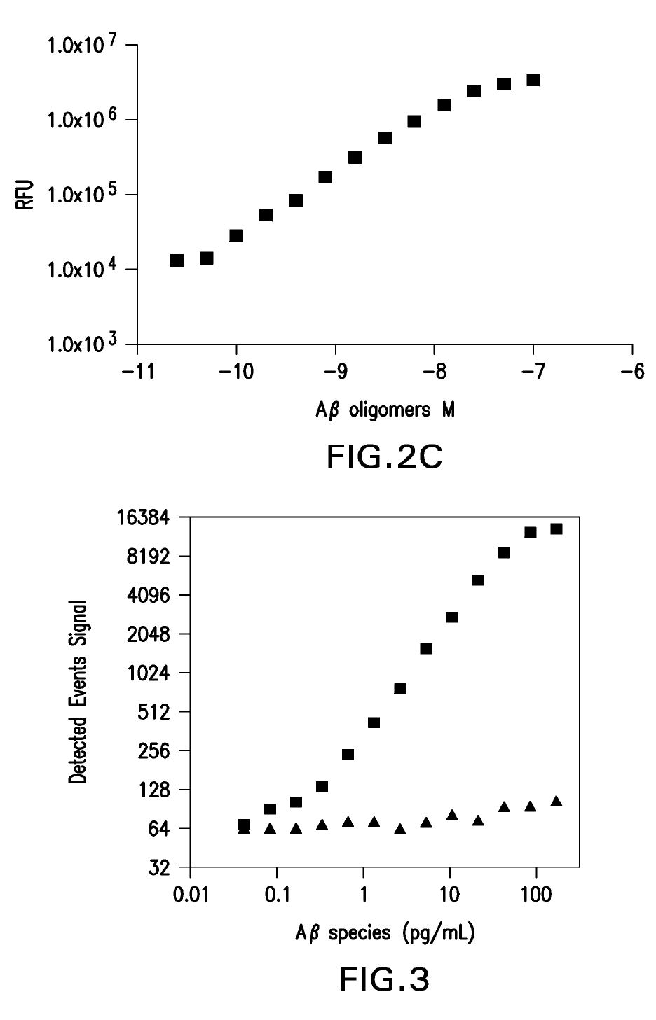

The 19.3 antibody was evaluated as a potential capture reagent for Aβ oligomers in combination with three different antibodies as detection antibodies 19.3, 7305 (U.S. Pat. No. 7,780,963, which is incorporated herein by reference in its entirety), and 82E1, following their biotinylation, in a sandwich ELISA format. Biotinylated 19.3 was examined as a detection antibody and paired with 19.3 as the capture antibody, in a test of overlapping epitopes. The presence of overlapping epitopes would be indicative of an Aβ construct with multiple epitopes, which suggests the presence of a dimer or higher order Aβ oligomers. The 19.3×19.3 overlapping epitope ELISA had a limit of detection (LoD) for Aβ oligomers of 98 pg/mL (FIG. 2C). Sandwich ELISAs for the antibody pair 19.3 and 82E1 (“19.3×82E1 sandwich ELISA”) (FIG. 2A), as well as the 19.3×7305 sandwich ELISA (data not shown), (LoD) of 1.3 pg/mL, a limit of reliable quantification (LoRQ) of 4.2 pg/mL for Aβ oligomers and the ratio of signal from Aβ oligomers/Aβ monomer was approximately 1,000:1, showing that the assay was 1,000 fold more selective for Aβ oligomers over Aβ40 monomer. Applicants found that the non-overlapping epitope assay, i.e. the 19.3×82E1 sandwich ELISA, was more sensitive as compared to recently published results for a similar assay employing the commercial Aβ antibody 6E 10 (FIG. 2B), which resulted in a limit of detection for Aβ oligomers of 98 pg/mL (Covance, Princeton, N.J.) (Gandy, et al., 2010, Ann. Neurol., 68:220-230) and equally sensitive as compared to the overlapping epitope assay employing the commercial antibody 82E1 (Xia, et al. 2009, Arch. Neurol., 66:190-199) (Immunobiological Laboratories, Inc., Minneapolis, Minn.). While the sandwich ELISAs carried out using chemiluminescence detection (FIGS. 2A, 2B, and 2C) were sufficient to detect Aβ oligomer standards, previous reports of CSF Aβ oligomer levels in the fM (fg/mL) range suggested that a selective ELISA-based Aβ oligomer assay would require ten to one hundred fold greater sensitivity levels to reliably detect and quantify Aβ oligomers in a CSF sample.

To increased the sensitivity of the sandwich ELISA assay, Applicants evaluated the performance of two antibody pairs in a paramagnetic micro-particle detection system, specifically the Erenna® system (Singulex®, Almeda, Calif.), employing detection of a fluorescent tagged detecting antibody that is uncoupled from the sandwich ELISA complex. Performance of the 19.3×82E1 sandwich ELISA was improved such that the 19.3×82E1 antibody pair enabled detection of Aβ oligomer signals in AD CSF samples at higher levels compared to either age-matched or younger control samples. More specifically, the assay LoD improved approximately thirty fold, to 0.04 pg/mL, while the LoRQ improved ten fold, to 0.42 pg/mL. Similarly, the Aβ oligomer/Aβ monomer ratio was also improved, to 5,000:1. As measured with this assay, the AD CSF samples had reduced Aβ42 levels and unchanged Aβ40 levels that were characteristic of AD patients. Taken together, the 19.3×82E1 sandwich ELISA using a paramagnetic micro-particle detection system, was able to reliably and specifically measure Aβ oligomer species in human CSF.

The term “Aβ oligomers” as used herein refers to multimer species of Aβ monomer that result from self-association of monomeric species. Aβ oligomers are predominantly multimers of Aβ42, although Aβ oligomers of Aβ40 have been reported. Aβ oligomers may comprise a dynamic range of dimers, trimers, tetramers and higher-order species following aggregation of synthetic Aβ monomers in vitro or following isolation/extraction of Aβ species from human brain or body fluids. ADDLs are one species of Aβ oligomers.

The term “neuronally derived protein” or “neuronally derived protein of interest” as used herein refers to a protein that is generated in and/or by the neurons in the brain that is to be measured by the inventive assays herein. In one embodiment of the invention herein, the neuronally derived protein is an Aβ oligomer that is present in the cerebrospinal fluid (CSF) sample of a human. This protein is distinguished from other Aβ oligomers that may be formed from Aβ in cells or tissue other than neurons.

The term “ADDLs” or “amyloid-β derived diffusible ligands” or “amyloid-β derived dementing ligands” as used herein refers to a neurotoxic, soluble, globular, non-fibrillar oligomeric structure comprising two or more Aβ protein monomers. Higher order oligomeric structures can be obtained not only from Aβ42, but also from any AP protein capable of stably forming the soluble non-fibrillar Aβ oligomeric structures, such as Aβ43 or Aβ40. U.S. Pat. No. 6,218,506 and WO 01/10900.

The term “Aβ fibrils” or “fibrils” or “fibrillar amyloid” as used herein refers to insoluble species of Aβ that are detected in human and transgenic mouse brain tissue because of their birefringence with dyes such as thioflavin S. Aβ species that form fiber-like structures comprised of Aβ monomers include β-pleated sheets. These species are believed to be immediate precursors to the extracellular amyloid plaque structures found in AD brain. The term “Aβ40 monomer” or “Aβ42 monomer” as used herein refers to the direct product of the enzymatic cleavage, i.e. aspartic protease activity, by β-secretase and γ-secretase on the amyloid protein precursor (APP) in a cell-free or cellular environment. Cleavage of APP by β-secretase generates the Aβ species beginning at Asp 1 (numbering as to Aβ peptide sequence after cleavage), while γ-secretase liberate the C-terminus of Aβ, predominantly either at residues 40 or 42.

The term “capture antibody” or “Aβ oligomer capture antibody” or “anti-human IgG2 capture antibody” as used herein refers to an antibody that is used as the capture antibody in the assays herein. The capture antibody as used herein binds to an Aβ oligomer or Aβ oligomer/antibody complex that are being measured and/or detected in the fluid sample. In one embodiment of the invention the capture antibody is the anti-Aβ oligomer antibody 19.3 and the complex detected is 19.3/Aβ oligomers. In another embodiment the capture antibody is an anti-human IgG2 capture antibody and the complex detected is IgG2/19.3/Aβ oligomers.

The term “IgG” or “IgG2” as used herein refers to any protein that functions as an antibody molecule. Each IgG is composed of four peptide chains—two heavy chains γ and two light chains. Each IgG has two antigen binding sites. There are four IgG subclasses (IgG1, 2, 3, and 4) in humans, named in order of their abundance in serum (IgG1 being the most abundant). The structure of the hinge regions gives each of the four IgG classes its unique biological profile.

The term “kappa light chain” as used herein refers to the portion of the Immunoglobulin G (IgG) that contains both an antigen binding domain and a constant region. There are two light chains per antibody molecule, which can be either of the kappa or lamba type, encoded on chromosomes 2 or 22, respectively. Two kappa light chains would be produced within B-cells, along with two heavy chains, assembled via disulfide bonds to form a complete IgG antibody molecule, and secreted to function as part of humoral immune defense system.

The term “biological sample” or “fluid sample” as used herein refers to any type of fluid, as compared to a tissue, or a vertebrate. Typical examples that may be used in the assays herein are blood, urine, tears, saliva, and cerebrospinal fluid, which is used in one embodiment of the invention. All other kinds of body fluids may also be used if Aβ oligomers are present.

The term “Alzheimer's disease” or “AD” or “amyloidogenic disorder” as used herein refers to the spectrum of dementias or cognitive impairment resulting from neuronal degradation associated with the formation or deposition of Aβ plaques or neurofibrillar tangles in the brain from the spectrum of diseases, including but not limited to, Down's Syndrome, Lewy body dementia, Parkinson's disease, preclinical Alzheimer's disease, mild cognitive impairment due to Alzheimer's disease, early onset Alzheimer's disease (EOD), familial Alzheimer's disease (FAD), thru the advance cognitive impairment of dementia due to Alzheimer's disease (Jack, et al., 2011, Alzheimer's Dement., May 7 (3):257-262), and diseases associated with the presence of the ApoE4 allele.

The term “limit of detection” of “LoD” as used herein refers to the sensitivity of the assays at the lowest concentration that can be detected above a sample which is identical except for the absence of the Aβ oligomers. The signal in the absence of Aβ oligomers is defined as the “Background.” As used herein, the LoD for Aβ oligomers was defined as ≧3 standard deviations above the mean of the background.

The “lower limit of reliable quantification” or “LLoRQ” as used herein refers to the sensitivity of the assay in combination with the coefficient of variability to indicate the lowest concentration that can be reliably and reproducibly differentiated from background. This limit typically defines the practical working range of the assay at the low end of sensitivity and is the concentration that delivers a coefficient of variability of ≦20% across≧three measured values.

### Identification And Characterization of A Selective Anti-Aβ Oligomer Capture Antibody

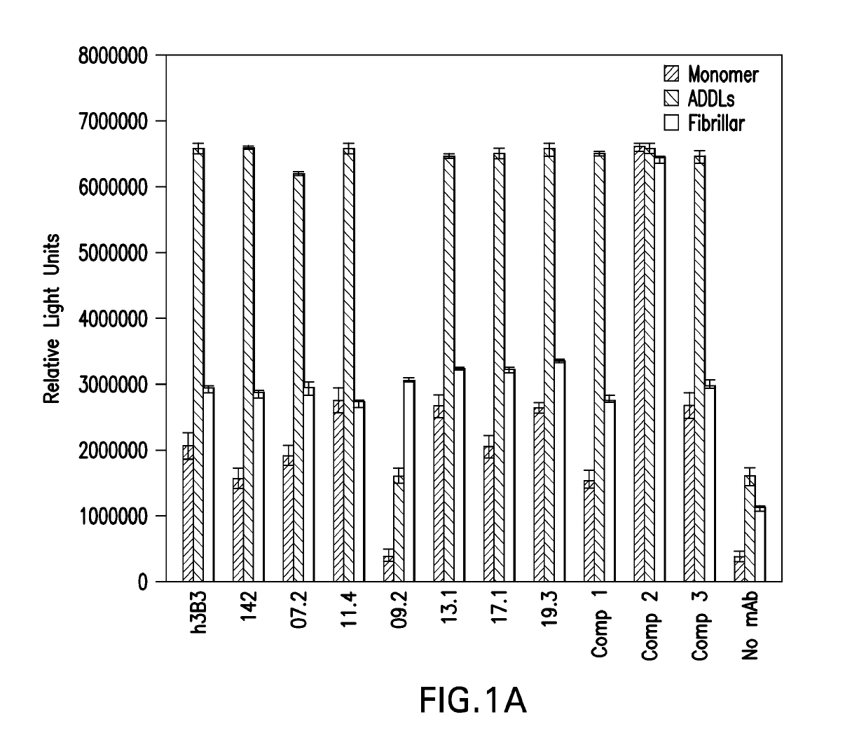

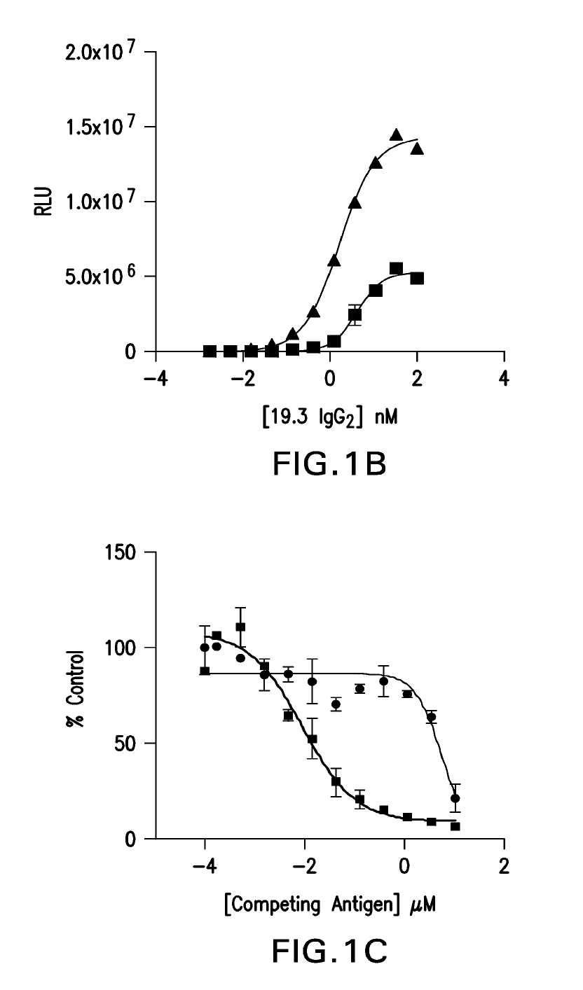

To develop an assay selective and specific for Aβ oligomers, Applicants first sought to identify an antibody that was both selective for and specific to ADDLs, a non-fibrillar species of Aβ oligomers. An anti-ADDL mouse monoclonal antibody, 3B3, was generated (U.S. Pat. Nos. 7,811,563 and 7,780,963) by immunizing mice with the ADDL Aβ oligomeric species mixed 1:1 with either Freund's (first and second vaccine, subcutaneously) or Incomplete Freunds Adjuvant (all subsequent vaccination, intraperitoneal). Each injection consisted of purified ADDLs equivalent to 194±25 μg total protein. The spleen from the mouse with the highest titer serum was fused with SP2/0 myeloma cells in the presence of polyethylene glycol and plated into 96-well plates. Cells were cultured at 37° C. with 5% CO₂ for 10 days in 200 μL of hypoxanthine-aminopterin-thymidine (HAT) selection medium. The cultures were fed once with Iscove's Modified Dulbecco's Medium (IMDM), (Sigma-Aldrich, St. Louis, Mo.), supplemented with 10% fetal bovine serum (FBS) on day 10, and the culture supernatants were removed on day 14 to screen for positive, Aβ oligomers antibody-containing, wells using a one-sided ELISA (Example?). The antibody 3B3 was selected for further development based on its ability to preferentially bind ADDLs as compared to Aβ monomer or Aβ fibrils (FIG. 1A).

The mouse clone 11/3B3 was converted to a human IgG2 antibody and designated as 19.3. The variable heavy and light chain domain regions of 3B3 encoding the Aβ oligomer binding domain were sequenced and cDNA generated encoding these CDRs were introduced in a human IgG2 context. An affinity maturation library was generated with the variable heavy and light chain domains of 3B3 introduced within the pFab3D phage display vector. The ligation products were transfected into E. coli TG1 cells and phage culture supernatant produced was titered, concentrated and aliquots made for phage library panning. Phage library panning was then conducted using biotinylated Aβ oligomers. The phages bound to biotinylated Aβ oligomers were eluted and added again to E. coli TG1 cells. Biotinylated Aβ oligomers were prepared using the same methods (Example 1) as the Aβ oligomers, but starting with N-terminal biotinylated Aβ42 peptide (American Peptide, Sunnyvale, Calif.). Phage supernatants (about 100 μl) were directly used for analysis in the Aβ monomer, Aβ oligomer, and Aβ fibril differential binding ELISA described above.

The anti-Aβ oligomer antibody 19.3, generated from the light chain affinity maturation library of 3B3, has been described and characterized in co-pending application PCT/US2011/______, claiming priority to 61/364,210, filed Jul. 14, 2010, and as used herein is an isolated antibody comprising:

a light chain variable region having the sequence
(SEQ ID NO: 1)
Ala Ser Arg Asp Val Val Met Thr Gln Ser Pro Leu
Ser Leu Pro Val Thr Pro Gly Glu Pro Ala Ser Ile
Ser Cys Arg Ser Ser Gln Ser Ile Val His Ser Asn
Gly Asn Thr Tyr Leu Glu Trp Tyr Leu Gln Lys Pro
Gly Gln Ser Pro Gln Leu Leu Ile Tyr Lys Ala Ser
Asn Arg Phe Ser Gly Val Pro Asp Arg Phe Ser Gly
Ser Gly Ser Gly Thr Asp Phe Thr Leu Lys Ile Ser
Arg Val Glu Ala Glu Asp Val Gly Val Tyr Tyr Cys
Phe Gln Gly Ser Arg Leu Gly Pro Ser Phe Gly Gln
Gly Thr Lys Leu Glu Ile Lys;
a heavy chain variable region having the sequence
(SEQ ID NO: 2)
Glu Val Gln Leu Val Glu Ser Gly Gly Gly Leu Val
Gln Pro Gly Gly Ser Leu Arg Leu Ser Cys Ala Ala
Ser Gly Phe Thr Phe Ser Ser Phe Gly Met His Trp
Val Arg Gln Ala Pro Gly Lys Gly Leu Glu Trp Val
Ala Tyr Ile Ser Arg Gly Ser Ser Thr Ile Tyr Tyr
Ala Asp Thr Val Lys Gly Arg Phe Thr Ile Ser Arg
Asp Asn Ala Lys Asn Ser Leu Tyr Leu Gln Met Asn
Ser Leu Arg Ala Glu Asp Thr Ala Val Tyr Tyr Cys
Ala Arg Gly Ile Thr Thr Ala Leu Asp Tyr Trp Gly
Gln Gly Thr Leu Val Thr Val Ser Ser;
and
a heavy chain constant region having the sequence
(SEQ ID NO: 3)
Ala Ser Thr Lys Gly Pro Ser Val Phe Pro Leu Ala
Pro Cys Ser Arg Ser Thr Ser Glu Ser Thr Ala Ala
Leu Gly Cys Leu Val Lys Asp Tyr Phe Pro Glu Pro
Val Thr Val Ser Trp Asn Ser Gly Ala Leu Thr Ser
Gly Val His Thr Phe Pro Ala Val Leu Gin Ser Ser
Gly Leu Tyr Ser Leu Ser Ser Val Val Thr Val Pro
Ser Ser Asn Phe Gly Thr Gln Thr Tyr Thr Cys Asn
Val Asp His Lys Pro Ser Asn Thr Lys Val Asp Lys
Thr Val Glu Arg Lys Cys Cys Val Glu Cys Pro Pro
Cys Pro Ala Pro Pro Val Ala Gly Pro Ser Val Phe
Leu Phe Pro Pro Lys Pro Lys Asp Thr Leu Met Ile
Ser Arg Thr Pro Glu Val Thr Cys Val Val Val Asp
Val Ser His Glu Asp Pro Glu Val Gln Phe Asn Trp
Tyr Val Asp Gly Val Glu Val His Asn Ala Lys Thr
Lys Pro Arg Glu Glu Gln Phe Asn Ser Thr Phe Arg
Val Val Ser Val Leu Thr Val Val His Gln Asp Trp
Leu Asn Gly Lys Glu Tyr Lys Cys Lys Val Ser Asn
Lys Gly Leu Pro Ala Pro Ile Glu Lys Thr Ile Ser
Lys Thr Lys Gly Gln Pro Arg Glu Pro Gln Val Tyr
Thr Leu Pro Pro Ser Arg Glu Glu Met Thr Lys Asn
Gln Val Ser Leu Thr Cys Leu Val Lys Gly Phe Tyr
Pro Ser Asp Ile Ala Val Glu Trp Glu Ser Asn Gly
Gln Pro Glu Asn Asn Tyr Lys Thr Thr Pro Pro Met
Leu Asp Ser Asp Gly Ser Phe Phe Leu Tyr Ser Lys
Leu Thr Val Asp Lys Ser Arg Trp Gln Gln Gly Asn
Val Phe Ser Cys Ser Val Met His Glu Ala Leu His
Asn His Tyr Thr Gln Lys Ser Leu Ser Leu Ser Pro
Gly Lys.

### Aβ Oligomer Selectivity of Anti-Aβ Oligomer Antibody 19.3

To confirm the binding potency of 19.3 for Aβ oligomers, as compared to Aβ40 monomer, one-sided ELISAs were completed using separate Aβ oligomers or Aβ40 monomer-coated plates with a common titration curve of the antibody (FIG. 1B). The EC50, a measure of the half-maximal total Aβ oligomer binding, of 19.3 was 1.6 nM and 4.3 nM for Aβ oligomers and Aβ40, respectively. In this format the 19.3 antibody demonstrated approximately three fold greater maximum binding for Aβ oligomers as compared to Aβ40 monomer, while the potency was approximately 3.7 fold greater. As shown in FIG. 1B, 19.3 had a greater affinity for Aβ oligomers versus Aβ40 monomer when both are independently immobilized on a assay plate surface. Thus, while the anti-Aβ oligomer antibody 19.3 identified herein selectively binds Aβ oligomers over Aβ40 monomer when each is bound independently to an assay plate, Applicants sought to further compare the relative binding properties of 19.3 when both Aβ oligomers and Aβ monomer species were present concurrently, such as that which would occur in a body fluid or tissue sample, either in solution or immobilized on an assay plate.

To more accurately represent an in vivo CSF sample, where both Aβ oligomers and Aβ monomers would be present, the affinity of 19.3 for Aβ oligomers in the presence of Aβ40 monomer was tested in a competitive ELISA format (FIG. 1C). The ELISA plate was prepared by first coating with a preparation of Aβ oligomers at 50 pmol per well and then adding the 19.3 antibody at a final concentration of 2 nM to each well. This concentration of 19.3, i.e. 2 nM, represents the EC50 concentration for Aβ oligomers binding determined in the one-sided ELISA (FIG. 1B). Adding Aβ40 monomer in a titration curve to competitively remove 19.3 from the Aβ oligomer-coated surface resulted in an EC50 of 5.5 μM. When 100 pmol per well of Aβ40 monomer was used to coat the ELISA plate and Aβ oligomers were used to compete for antibody binding, the EC50 was 8.7 nM. This indicated that 19.3 had higher affinity for Aβ oligomers, both in solution and in a solid phase, as compared to Aβ40 monomer. Accordingly, the concentration of Aβ40 required to displace 50% of 19.3 from Aβ oligomers was approximately 600 fold higher than the concentration of Aβ oligomers required to displace 19.3 binding to Aβ40. Concentrations up to 0.200 pM of Aβ oligomers have been reported in CSF from AD patients (Georganopoulou, et al., 2005, Proc. Natl. Acad. Sci. U.S.A., 102:2273-2276) as compared to 1500 pM of Aβ monomer. Thus, the antibody 19.3 appeared to have the degree of selectivity that would be required to detect Aβ oligomers above background levels of Aβ monomer. The 19.3 antibody was coupled with a detecting antibody, 82E1, previously reported in ELISA formats to detect Aβ oligomers in AD brain (Xia, et al., 2009, Arch. Neurol., 66:190-199) for further assay development. When 82E1 (Immunobiological Laboratories (IBL), Inc., Minneapolis, Minn.) was used as both the capture and detection antibody, 82E1/82E1 ELISA, this antibody had selectivity below the level required for use with human CSF (data not shown).

### Aβ Oligomer Preferring Antibodies In Aβ Oligomer Sandwich ELISA

In a screen of capture and detecting antibody pairs in a sandwich ELISA format (Table 1), the combination of 19.3 as the capture antibody with either 7305, an anti-Aβ oligomer antibody (20C2, U.S. Pat. No. 7,780,963, which is incorporated herein by reference in its entirety) or 82E1 (Immunobiological Laboratories (IBL), Inc., Minneapolis, Minn.) performed comparably in Casein blocking buffer in an Aβ oligomer standard curve, each giving a limit of detection (LoD) under 4 pg/mL (FIG. 2A). Use of an anti-Aβ monomer antibody as both capture and detection antibody has been reported as an Aβ oligomer assay, however, absolute levels of sensitivity or selectivity were either not reported (6E10/6E10; Gandy, et al., 2010, Ann. Neurol., 68:220-230), or selectivity was below (82E1/82E1; Xia, et al., 2009, Arch. Neurol., 66:190-199) that desired for an assay to measure Aβ oligomers in human CSF.

While neither Gandy nor Xia have reported detection of Aβ oligomers in human CSF, Applicants internal work with 6E10 and reports published by IBL with 82E1 suggested that their sensitivity might be in the range needed for Aβ oligomer detection in human CSF. Applicants compared herein the use of identical antibodies for both capture and detection antibodies, such as 6E10/6E10 (FIG. 2B) and 19.3/19.3 (FIG. 2C), as well as sandwich ELISA assay pairs using 19.3 as a capture antibody only (FIG. 2A, with 82E1 detection). As shown in Table 1, 6E10/6E10 and 19.3/19.3 both demonstrated approximately one hundred fold reduced sensitivity compared to either 19.3/7305 or 19.3/82E1. The 19.3/82E1 ELISA utilizing luminescence detection technology (EnVision® Multilabel plate reader, PerkinElmer, Waltham, Mass.) (FIG. 2A), generated a LoD of approximately 1.3 pg/mL. In this assay format, the LLoRQ of Aβ oligomer was 4.2 pg/mL (with coefficients of variance less than 20% at this lowest measure) and the assay was approximately 1000 fold-selective for Aβ oligomer signal as compared to Aβ40 monomers. While this assay was used to evaluate Aβ oligomer preparations, it was not sensitive enough to reliably detect Aβ oligomer levels in human CSF at levels consistent with previous estimates (Georganopoulou, et al., 2005, Proc,. Natl. Acad. Sci. U.S.A., 102:2273-2276). The 19.3×82E1 sandwich ELISA was advanced into a paramagnetic micro-particle detection immunoassay platform that has been reported to have greater sensitivity to detect analytes in human body fluids (Erenna®, Singulex®, Alameda, Calif.).

TABLE 1

| | Capture Antibody | | |
|---|---|---|---|
| Detection Antibody | 19.3 | 7305 | 6E10 |
| 19.3 | 1 | | |
| 7305 | 2 | 2 | |
| 6E10 | | | 1 |
| 82E1 | ✓ | | |

1 reduced sensitivity compared to 19.3 x 82E1
2 unacceptable Background in human CSF (fibrinogen cross-reactivity)
3 reduced selectivity compared to 19.3 x 82E1

### Aβ Oligomer-Selective Sandwich ELISA With Improved Sensitivity

Both the 19.3 and 7305 (19.3×7305) and the 19.3 and 82E1 (19.3×82E1) antibody pairs (Table 1) were evaluated in a sandwich ELISA using a paramagnetic micro-particle detection immunoassay system, Erenna® Immunoassay System (Singulex®, Almeda, Calif.) to determine if assay sensitivity could improve further for the measurement of Aβ oligomers in human and non-human primate fluid samples. In one embodiment of the invention, the immunoassay was conducted using human CSF samples.

While paramagnetic micro-particle immunoassays, such as the Erenna® Immunoassay System, have been used for biomarkers present in a biological sample in the nanomolar (nM) range, such as Aβ40 and Aβ42, it has not been demonstrated prior to Applicants work herein that such an immunoassay system could specifically and reliably detect a biomarker present in a CSF sample in the femtomolar (fM) range, such as the Aβ oligomers herein. Without wishing to be bound by any theory, Applicants believe, and have demonstrated, that the specificity and sensitivity of the claimed assays is attributable to the specificity and sensitivity of the anti-ADDL antibody pair selected and used in the sandwich ELISA. Similarly, while Applicants have used the Erenna® Immunoassay System to illustrate the claimed assay, it is possible that other detection systems having comparable sensitivities could be employed in the inventive methods.

The 19.3×7305 sandwich ELISA was conducted using the Erenna® Immunoassay System (Singulex®, Almeda, Calif.), covalently-coupling the 19.3 antibody to the Erenna® micro-particle (MP) beads (hereinafter “19.3/MP beads”). The 19.3/MP beads were then mixed with buffer containing a standard curve of either Aβ oligomer or monomeric Aβ40. The resulting 19.3/MP bead/Aβ oligomer or Aβ40 complex (hereinafter “Aβ oligomer complex”) was washed and either a fluorescently-tagged 7305 or 82E1 detection antibody was bound to the Aβ oligomer complex. The Erenna® instrument, using a proprietary detection technology capable of single-molecule counting (see U.S. Pat. No. 7,572,640), measured the fluorescently-labeled detection antibody following its release from the sandwich ELISA. As shown in Table 2 data from the 19.3×7305 assay, using a two-fold dilution of the Aβ oligomer standard in buffer, aligned with a linear two-fold dilution of fluorescent signal (detected events mean). Signals generated by neat rhesus CSF, or CSF to which a standard curve of Aβ oligomers was introduced, demonstrated that the fluorescent signal attributed to binding of the tagged 7305 antibody was equivalent in both cases, while the 19.3×82E1 sandwich assay was able to detect spiked Aβ oligomers across the full standard curve. In the assay format using 7305 as the detection antibody, this was indicative that there was a non-specific background (from something present in the rhesus CSF) saturating over the range of the Aβ oligomers dilution series that was sufficient to detect Aβ oligomers in buffer alone. Subsequently, the fluorescent signal was found to be identical to that for a naked micro-particle, even in the absence of the 19.3 antibody coupling (data not shown), which was also consistent with a non-specific signal due to 7305 antibody cross-reactivity.

TABLE 2

| | Expected | | | | | Interp | | | |
|---|---|---|---|---|---|---|---|---|---|
| Standard | [ADDLs] | | | | | [ADDLs] | | | % |
| Diluent | pM | n | DE Mean | SD | CV % | pM Mean | SD | CV % | Recovery |
| Standards | 5.00 | 3 | 5579 | 506 | 9 | 5.1 | 0.5 | 10 | 103 |
| Diluent | 1.67 | 3 | 1942 | 235 | 12 | 1.7 | 0.2 | 13 | 100 |
| | 0.56 | 3 | 691 | 152 | 22 | 0.5 | 0.1 | 25 | 96 |
| | 0.19 | 3 | 324 | 43 | 13 | 0.2 | 0.1 | 17 | 116 |
| | 0.06 | 3 | 131 | 34 | 26 | 0.1 | 0.1 | 49 | 88 |
| | 0.00 | 3 | 72 | 28 | 39 | ND | | | |
| Rhesus CSF- | 5.00 | 3 | 9097 | 88 | 1 | | | | |
| Depleted | 1.67 | 3 | 9112 | 195 | 2 | | | | |
| | 0.56 | 3 | 8721 | 166 | 2 | | | | |
| | 0.19 | 3 | 8785 | 269 | 3 | | | | |
| | 0.06 | 3 | 8744 | 273 | 3 | | | | |
| | 0.00 | 3 | 8678 | 519 | 6 | | | | |
| Rhesus CSF- | 5.00 | 3 | 10353 | 237 | 2 | | | | |
| Non-Depleted | 1.67 | 3 | 9719 | 495 | 5 | | | | |
| | 0.56 | 3 | 9902 | 546 | 6 | | | | |
| | 0.19 | 3 | 9971 | 319 | 3 | | | | |
| | 0.06 | 3 | 9721 | 329 | 3 | | | | |
| | 0.00 | 3 | 10515 | 282 | 3 | | | | |

A second embodiment of the Aβ oligomer selective sandwich ELISA developed using the Erenna® Immunoassay System replaced the 7305 detection antibody with 82E1, also coupled to a fluorescent tag. As shown in Table 3, this embodiment of the assay eliminated the non-specific signal in both the neat and Aβ oligomer depleted rhesus CSF, further supporting the belief that the 7305 antibody had been the source of the non-specific signal. Without wishing to be bound by any theory, the high background signal observed for the 19.3/7305 antibody pair was believed to be due to CSF fibrinogen binding to the MP beads, which was not observed for the 19.3/82E1 antibody pair. This embodiment of the Aβ oligomer selective sandwich ELISA generated a LoD of the Aβ oligomer standards at 0.04 pg/mL, a LLoRQ at 0.42 pg/mL and 5,000 fold selectivity of the assay for Aβ oligomers over Aβ40 monomer (FIG. 3). On the basis of these findings, Applicants selected this assay format for further optimization.

TABLE 3

| | 19.3/7305 | 19.3/82E1 |
|---|---|---|
| Parameter | Antibody Pair | Antibody Pair |
| Slope detected events (pM) | 1,200 | 4,000 |
| Background | 70 | 100 |
| LoD (pM) | 0.01 | 0.01 |
| LLoRQ (pM) | 0.16-0.49 | 0.12 |
| Aβ40 monomer Cross Reactivity | 0.02% | 0.04% |
| Depleted Rhesus CSF (pM) | 80 | <0.12 |
| Non-Depleted Rhesus CSF (pM) | 200 | 0.35 |

### Pharmacodynamic (PD) Assay

Using the findings above, Applicants have developed an selective Aβ oligomer sandwich ELISA, using the 19.3 and 82E1 antibody pair, to detect and measure the levels of Aβ oligomers in a CSF sample. This assay will heretofore be called the pharmacodynamic (PD) assay for its use to assess changes in the analyte, i.e. Aβ oligomer, levels (FIG. 9A) following treatment to inhibit production, increase clearance, or otherwise modify Aβ oligomer levels. The PD assay can also be used to differentiate AD from non-AD patients, i.e. diagnostic, to monitor the progression of the disease, i.e. prognostic, or to monitor the therapeutic potential of a disease-modifying treatment to change Aβ oligomer concentrations.

The PD assay, as described in Example 7, placed the 19.3 antibody coupled to a paramagnetic micro-particle (MP) bead (MP bead/19.3) into a well on an ELISA plate. To the well was added either a human CSF or an Aβ oligomer standard (in a dilution series added to a Tris buffer and bovine serum albumin). Any Aβ oligomer present in the well was bound by the 19.3/MP bead and the excess solution was washed away. Fluorescent-labeled 82E1, as the detection antibody, within an assay buffer (Tris buffer with 1% triton X-100, d-desthiobiotin, BSA), was added to the washed MP bead/19.3/Aβ oligomer complex and incubated, to bind the Aβ oligomer complex. The resulting MP bead/19.3/Aβ oligomer/82E1 complex was washed with an elution buffer and the fluorescent-labeled 82E1 antibody is eluted with any unbound antibody. Detection with the paramagnetic micro-particle detector, such as the Erenna® instrument, in which the solution flows by and is excited by a laser, allows the detection of single molecules (fluorescent tag emits photons of a specific light wavelength) to generate and measure a fluorescent signal, equivalent to the molecules detected, i.e. Aβ oligomer. A standard curve of Aβ oligomers, as measured with the Erenna® instrument, as compared to Aβ monomers is shown in FIG. 3.

### Aβ Oligomers In Human CSF

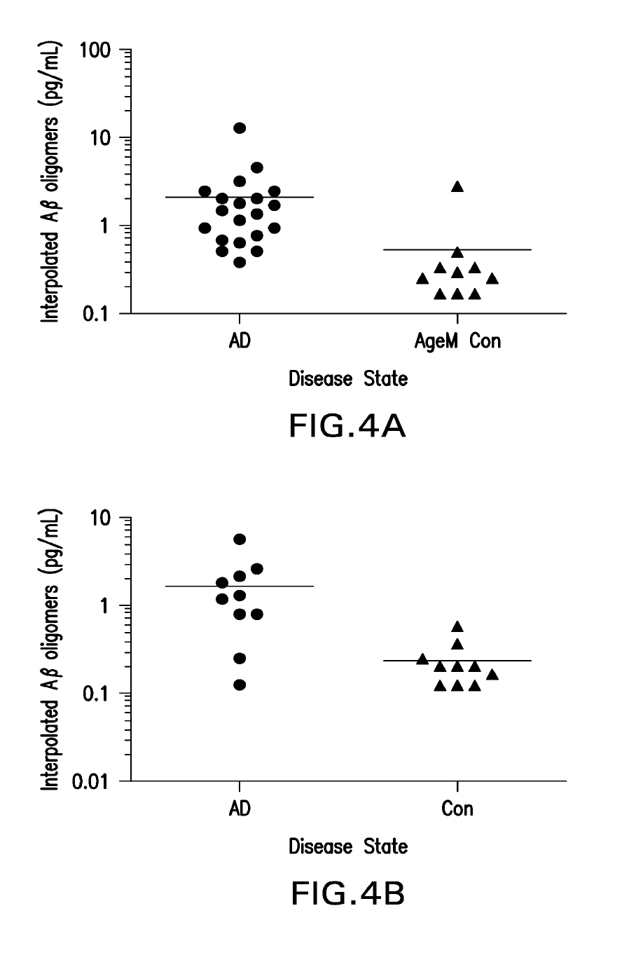

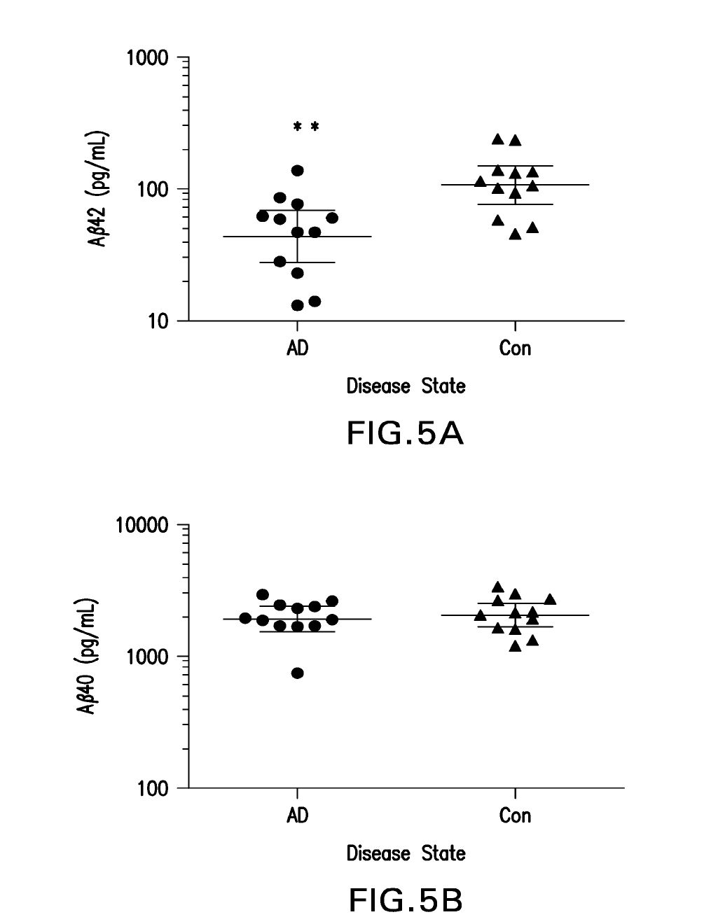

The 19.3×82E1 Aβ oligomer selective sandwich ELISA of Example 6 was used to measure endogenous levels of Aβ oligomers in human CSF samples (FIGS. 4A and 4B). In two separate sample cohorts, the fluorescent signal, generated by the presence of Aβ oligomers, was significantly elevated in AD (clinically diagnosed using a MMSE score below 25 as probable AD) CSF as compared to either young or healthy age matched controls. The absolute levels of Aβ oligomers observed were 2.1 +/−0.61 pg/mL in AD (N=20) and 0.53 +/−0.26 pg/mL in age-matched control (N=10) in the CSF samples from Precision Medicine (Solana Beach, Calif.) with a t-test, two way Mann-Whitney score of p<0.0004 (FIG. 4A). The absolute levels of Aβ oligomers observed were 1.66 +/−0.5 pg/mL in AD (N=10) and 0.24 +/−0.05 pg/mL in control (N=10) in the CSF samples from Bioreclamation (Hicksville, N.Y.), with a t-test, two way Mann-Whitney score of p<0.0021 (FIG. 4B). Combining the two cohorts, 90% of the diagnosed AD CSF samples were above the LLoRQ of 0.42 pg/mL, while only 20% of the age-matched control or 10% of the young controls were above this limit. All values were above the LoD of 0.04 pg/mL. Aβ40 and Aβ42 monomer levels were measured in the CSF samples obtained from Bioreclamation (FIGS. 5A and 5B, respectively) and were comparable between the AD and control CSF for Aβ40 (FIG. 5A), while they were significantly reduced in the AD samples for Aβ42 (FIG. 5B). This has been previously reported as a feature (De Meyer, et al., 2010, Arch. Neurol. 67:949-956; Jack, et al., 2010, Lancet Neurol. 9:119-128) of AD CSF and confirmed the correct diagnosis of these samples. Without wishing to be bound to any theory, Applicants believe that the lower levels of Aβ42 in the AD CSF samples is due to retention of Aβ42 in the amyloid deposits of the AD brain. The ability to specifically detect and quantify these observed differences suggests that these biomarkers can be used as a diagnostic and prognostic measure for AD.

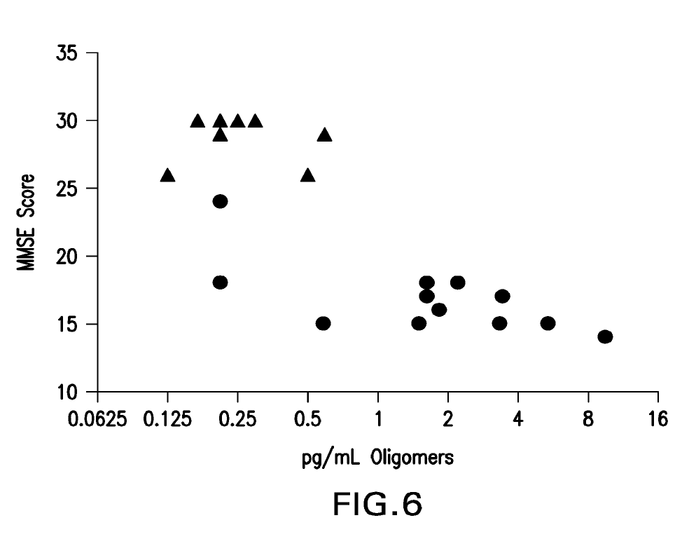

For a diagnostic assay, the signal, i.e. the level of Aβ oligomers, detected from the inventive assay herein would typically be greater than three fold higher for an AD patient (to a level >0.5 pg./mL) as compared to the signal observed for non-AD patients. This is consistent with the data shown in both FIG. 4A, in which the levels of Aβ oligomers in the AD CSF compared to age-matched controls was four-fold higher and in FIG. 4B, in which levels of Aβ oligomers in AD CSF was eight-fold higher. This data also supports the use of the inventive Aβ oligomer assay to identify patients at early stages of disease (i.e., a prognostic assay). Age is the biggest risk factor for the development of AD and the differences observed between AD and age-matched controls were smaller than between AD and young controls. Similarly, for a prognostic Aβ oligomer assay, patients having a MMSE of below 25 would have a detected Aβ oligomer signal of ≧0.5 pg/mL (four to eight fold higher than patients with MMSE above 25/normal) as compared to the signal detected for Aβ42 monomer, which is approximately two-fold lower in the AD CSF compared to controls. FIG. 6 suggests that if an MMSE score of ≦25 is used as a cutoff (Mungas, D., 1991, Geriatrics 46 (7): 54-58), above which a patient is considered ‘normal healthy’ and below which a patient is considered as either mildly cognitively impaired, or as having AD, it would be expected that above an Aβ oligomer level of 0.5 pg/mL, the patient would be likely to have an MMSE score below 25.

### Target Engagement (TE) Assay

Similarly, using the findings above, Applicants have developed a selective sandwich ELISA, using an anti human IgG2 antibody×82E1 antibody pair, to detect and quantify levels of bound Aβ oligomers in a CSF sample from a patient treated with the anti-Aβ oligomer 19.3, IgG2, antibody, i.e. a therapeutic antibody. This assay will heretofore be called the target engagement assay (TE Assay) for its use to measure Aβ oligomers bound in vivo to a therapeutic (capture) antibody. As such, the TE assay can be used to measure levels of Aβ oligomers bound to a therapeutic antibody to confirm engagement of the Aβ oligomer by the therapeutic. Without wishing to be bound by any theory, Applicants believe that the level of Aβ oligomers bound to a therapeutic anti-Aβ oligomer antibody will be lower in CSF samples from subjects who have been treated over time with said therapeutic. Levels of bound Aβ oligomers that increase or are unchanged post-administration would suggest that the therapeutic is not suitable for the treatment of AD. Alternatively, it may be the case that merely by sequestering the Aβ oligomers and binding them to the therapeutic antibody, a benefit may be obtained in acute performance, due to reduced interaction with neurons in the brain. However, this benefit may not be associated with a change in Aβ oligomers per se. The target engagement assay would assess, at a minimum, the ability of a therapeutic antibody to engage Aβ oligomers within the CSF.

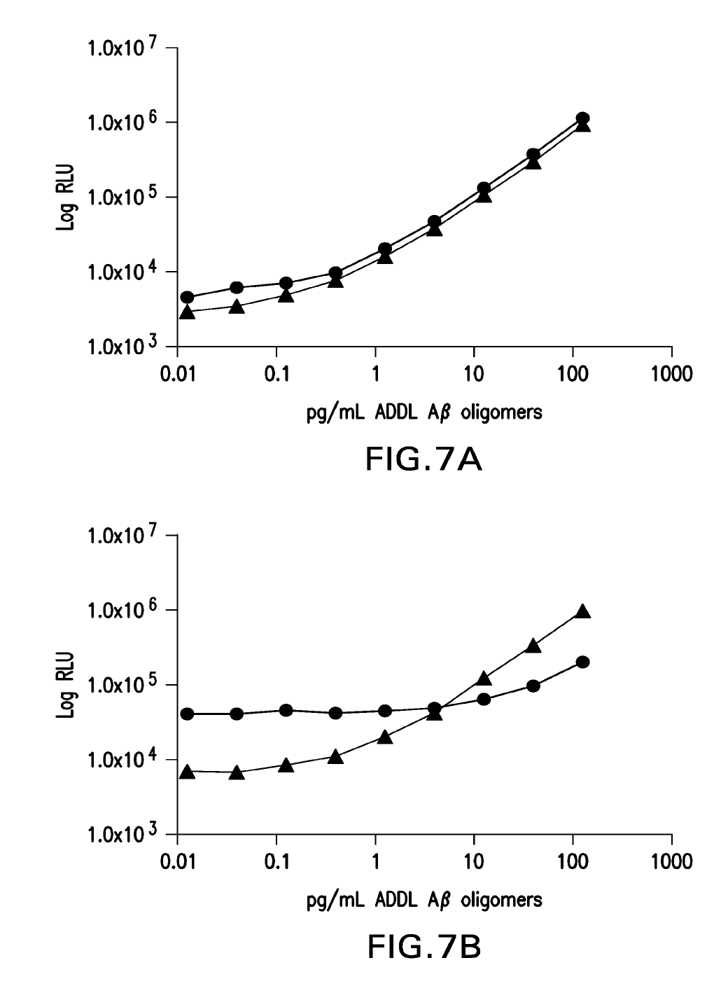

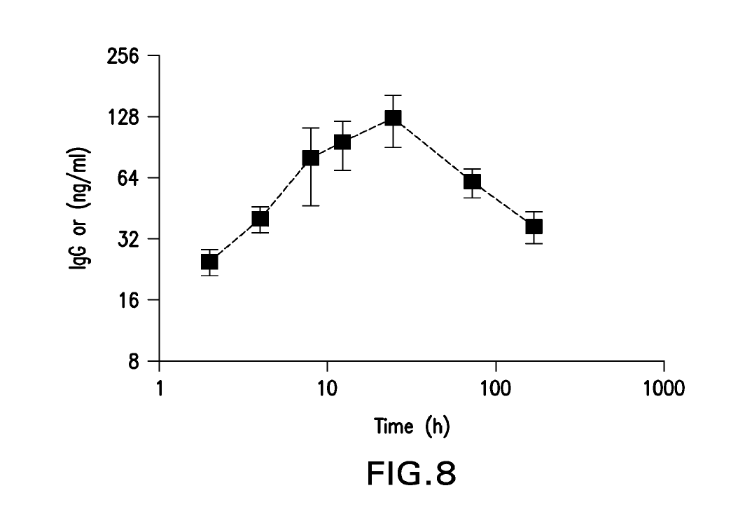

To demonstrate the ability of Aβ oligomer-specific antibodies to engage Aβ oligomers in a human CSF matrix, Applicants generated 19.3/Aβ oligomers complexes within human CSF by spiking in the anti-Aβ oligomer antibody 19.3 to levels believed to be present at 24 hours in rhesus monkey dosed N with 20 m/k (100 ng/mL, FIG. 8). To this 19.3-spiked human CSF sample was added an escalating amount of Aβ oligomer standards, both matching endogenous Aβ oligomer concentrations (0.1-5.0 pg/mL) (FIGS. 4A and 4B) and also raising them significantly above normal ranges. The 19.3×Aβ oligomer complexes formed in human CSF were captured onto 96-well ELISA plates coated with either antibody to human kappa light chain or antibody to human IgG2, then detected with biotinylated 82E1 (b82E1) as was done for the PD assay (FIG. 3A). The anti-Aβ oligomer antibody 19.3 was sufficiently recognized by both anti-human kappa and anti-human IgG2 in buffer (▴, FIGS. 7A and 7B), as the antibody contains both of these features. As shown in FIG. 7A (, CSF), the assay using anti-human IgG2 as the capture antibody and 82E1 as the detection antibody, to detect and measure the 19.3/Aβ oligomer complex, resulted in significantly better sensitivity in human CSF as compared to the assay using anti-human kappa as the capture antibody (, CSF, FIG. 7B). Both assays had equivalent sensitivity in a Casein buffer. Use of anti-human kappa to capture the 19.3/Aβ oligomer complex resulted in less sensitivity, to a LoD of 42 pg/mL Aβ oligomer bound to 100 ng/mL 19.3, perhaps due to higher background levels of IgG species with a kappa light chain in human CSF as compared to IgG2 species, which resulted in greater sensitivity for the assay format using an anti-IgG2. Following dosing of either human or experimental animals with 19.3 or a related IgG2 anti-Aβ oligomer antibody as a therapeutic antibody, one would expect the therapeutic antibody to be represented in the CSF at 0.1-0.2% of the dosed level (Thompson, 2005, Proteins of the Cerebrospinal Fluid, Elsevier Academic Press, New York, N.Y.). The therapeutic antibody present in the CSF would be bound to available Aβ oligomers, the 19.3 (IgG2)/Aβ oligomer complexes would be captured with the anti-IgG2 capture antibody through the anti-human 19.3, IgG2, antibody, and the Aβ oligomer complexes would then be detected with 82E1. The sensitivity of this platform would likely improve using a paramagnetic micro-particle detection system, such as the Erenna® immunoassay system (Singulex®, Alameda, Calif.), utilized in the PD assay above.

Over time, following therapeutic treatment with an anti-Aβ oligomer antibody, it is expected that the signal detected for the 19.3/Aβ-oligomer complexes would be reduced (as compared to pre-treatment levels). The amount of bound Aβ oligomer, whether as measured for these complexes acutely or after a period of therapeutic treatment, represents the proportion of the therapeutic antibody engaged with the target, i.e. Aβ oligomers, and could serve as a surrogate for the efficacy of the therapeutic antibody.

### EXAMPLES

The following abbreviations are used herein: Ab: antibody; Aβ: amyloid beta protein; AD: Alzheimer's disease; ADDLs: amyloid-βderived diffusible ligands; aM: attomolar; CSF: cerebrospinal fluid; DE mean: detected events mean; DMSO: dimethylsulfoxide; HFIP: 1,1,1,3,3,3 hexafluoro-2-propanol; HMW: high molecular weight; LMW: low molecular weight; LoD: limit of detection; LLoRQ: lower limit of reliable quantification.

### Example 1

### ADDL Preparations And Aβ

Aβ40 and Aβ42 (amyloid β peptide 1-40, amyloid β peptide 1-42) were obtained from the American Peptide Co., Sunnyvale, Calif. Aβ42 was dissolved in 1,1,1,3,3,3 hexafluoro-2-propanol (HFIP), Sigma-Aldrich, St. Louis, Mo., to eliminate any pre-existing secondary structure that could act as a “seeds” for aggregation. The HFIP was removed by evaporation to form an Aβ42 film. The Aβ42 peptide film (1 mg Aβ42 dried down from 100% HFIP solvent) was dissolved in 44 μL of DMSO, to which 1,956 μl of cold F12 media (GIBCO®, Invitrogen, Carlsbad, Calif., Cat #ME100014L1) was added with gentle mixing and incubated at room temperature for 18 to24 hours. Samples were centrifuged at 14,200 g for 10 minutes at room temperature. Supernatent was transferred to a fresh tube and was filtered through 0.5 ml column YM-50 filter tube (Millipore, Bedford Mass.; Cat #UFC505096, 0.5 ml) via spin at 4,000 rpm for 15 minutes at 4° C. The retentate was collected by reversing the filter insert, replaced into a new collection tube, and centrifuged at 4,000 rpm for 5 minutes at 4° C. Protein concentration was measured pre-filtration by Bradford Assay (BioRad, Hercules, Calif., Cat #_{—}23236) and reported as μM (calculated based on Aβ monomer molecular weight (MW 4513)). All samples were stored at −80° C. until used.

### Example 2

### Selection of Anti-ADDL Antibodies

### A. Panning Humanized Antibody Library

An affinity mature library of a humanized anti-ADDL antibody, h3B3, (See U.S. Pat. Nos. 7,811,563 and 7,780,963) was constructed in which part of the light chain CDR3 amino acid sequences were subject to random mutagenesis. To cover the entire CDR3 region, two sub-libraries were built. One library was composed of the parental heavy chain variable region and mutated amino acids in the left half of the light chain CDR3 and the other in the right half of the light chain CDR3. A similar strategy was used for heavy chain CDRs random mutagenesis with three sub-libraries.

Humanized 3B3 (h3B3) was subjected to affinity maturation using methods known in the art. The h3B3 variable regions were cloned in a Fab display vector (pFab3D). In this vector, the variable regions for heavy and light chains were in-frame inserted to match the CH1 domain of the constant region and the kappa constant region, respectively. In Fab3D, myc epitope and six consecutive histidine amino acids follow the CH1 sequence, which is then linked to the phage pill protein for display. All positions in the heavy and light chain CDR3s were randomly mutagenized using degenerate oligonucleotide sequences built in the PCR primers. To accommodate the physical size, the sub-libraries were constructed with each focusing on 5-6 amino acids. The vector DNA of human 3B3 (H3B3) was used as template DNA to amplify both heavy and light chains with the mutated PCR primers (Table 4). After PCR amplification, the synthesized DNA fragments were run on a 1.3% agarose gel, the primers removed and the variable fragments digested with restriction enzymes: BsiWI and XbaI cloning sites for light chain variable cloning, XhoI and ApaI for heavy chain variable cloning.

TABLE 4

| 3B3 Affinity | Forward | Reverse |
|---|---|---|
| Maturation Library | PCR Primer | PCR Primers |
| Light Chain | SEQ ID NO: 4 | SEQ ID NO: 5 |
| Libraries | | SEQ ID NO: 6 |
| Heavy Chain | SEQ ID NO: 7 | SEQ ID NO: 8 |
| Libraries | | SEQ ID NO: 9 |

To construct an affinity maturation library in pFab3D phage display vector, pFab3D-3B3 DNA was digested with the same pair of the restriction enzymes, purified and the PCR fragments for heavy or light chain variables ligated with T4 ligase (Invitrogen, Carlsbad, Calif.) overnight at 16° C. The ligation products were then transfected into E. coli TG1 electroporation-competent cells (Stratagene, Agilent Technologies, Santa Clara, Calif.) and aliquots of the bacterial culture plated on LB agar-carbenicillin (50 μg/mL) plates to titer library size. The remaining cultures were either plated on a large plate with carbenicillin and incubated at 30° C. overnight for E. coli library stock or infected with helper phage M13K07 (Invitrogen, Carlsbad, Calif., 10¹¹ pfu/mL) by incubating at room temperature and 37° C. for ten minutes. Then 2TY medium with carbenicillin (50 μg/mL) was added and incubated at 37° C. for one hour with shaking Kanamycin (70 μg/ml) was then added and the cultures grown overnight at 30° C. with shaking. The phage culture supernatant was tittered and concentrated by precipitation with 20% (v/v) PEG (polyethleneglycol)/NaCl, resuspended in PBS, sterilized with a 0.22 μm filter, and aliquots made for phage library panning.

The phage library panning was then conducted as summarized in Table 5.

TABLE 5

| Panning Rounds | Round 1 | Round 2 | Round 3 | Round 4 |
|---|---|---|---|---|
| Antigen | 180 nM | 60 nM | 20 nM | 10 nM |
| concentration | | | | |

Input phages from the Fab display phage libraries (100 μl, about 10¹¹⁻¹²pfu) were blocked with 900 μL of blocking solution (3% non-fat dry milk in PBS) to reduce nonspecific binding to the phage surface. Streptavidin-coated beads were prepared by collecting 200 μL of the bead suspension in a magnetic separator and removing supernatants. The beads were then suspended in 1 mL of blocking solution and put on a rotary mixer for 30 minutes. To remove non-specific Streptavidin binding phage the blocked phage library was mixed with the blocked streptavidin-coated beads and placed on a rotary mixer for thirty minutes. Phage suspensions from the de-selection process were transferred to a new tube and 200 μL of antigen, 10% bADDL was added and incubated for two hours for antibody and antigen binding. After the incubation, the mixture was added into the blocked Streptavidin-coated beads and incubated on a rotary mixer for one hour to capture the Ab/Ag complex on streptavidin beads. The beads with captured 10% bADDL/ phage complexes were washed five times with PBS/0.05% Tween 20 and then twice with PBS alone. The bound phages were eluted from the bADDL with 200 μL of 100 mM TEA and incubated for twenty minutes. The eluted phage were then transferred to a 50 mL tube, neutralized with 100 μL of 1M Tris-HCl, pH7.5, and added to 10 mL of E. coli TG1 cells with an OD 600 nm between 0.6-0.8. After incubation at 37° C. with shaking for one hour, culture aliquots were plated on LB agar-carbenicillin (50 μg/mL) plates to titer the output phage number, and the remaining bacteria centrifuged and suspended with 500 μl 2xYT medium (Teknova, Hollister, Calif.), plated on bioassay YT agar plates (Teknova, Hollister, Calif.) containing 100 μg/ml ampicillin and 1% glucose. The bioassay plates were grown overnight at 30° C.

After each round of panning, single colonies were randomly picked to produce phage in 96-well plates. The procedures for phage preparation in 96-well plate were similar to that described above except no phage precipitation step was used. Culture plates containing colonies growing in 120 μL of 2xYT medium (16 g Bacto-tryptone, 10 g Bacto-yeast extract, 5 g NaCl (all, BD Biosciences, Franklin Lakes, N.J.), ddH2O to 1 L (autoclave)) with 100 μg/ml ampicillin and 0.1% glucose were incubated overnight in a HiGro® shaker (Genomic Solutions, Ann Arbor, Mich.) at 30° C. with shaking at 450 rpm. The phage supernatants (about100 μL) were directly used for analysis in the AβDDL binding ELISA described above. Binding of phage to ADDLs was detected with an anti-M13-antibody conjugated to horseradish peroxidase (HRP) (Amersham Bioscience, GE Healthcare, Waukesha, Wis.).

### Example 3

### Determination 19.3 EC50 For Aβ Oligomers And Aβ40

High protein binding plates were coated at either 100 pmol/well Aβ40 or 50 pmol/well Aβ oligomers in PBS, overnight at 4° C. Next day, plates were washed five times with PBS+0.05% Tween-20 and blocked overnight with Casein blocking buffer (Thermo Scientific, Waltham, Mass.) and 0.05% Tween-20. The 19.3 antibody, identified in Example 2, was tested at 0 to 15 μg/ml in a 12-point three fold dilution series. After two hours at room temperature incubation, the plates were washed and alkaline phosphatase conjugated anti-human IgG (ThermoScientific, Waltham, Mass.) was added at 0.08 μg/ml. After incubation for 45 minutes at room temperature, the plates were washed and Tropix CDP star (Applied Biosystems, Foster City, Calif.) was added. Luminescence was detected after 30 minutes on an EnVision® plate reader (PerkinElmer, Waltham, Mass.). Curve fits were completed using GraphPad Prism (GraphPad Software, Inc., San Diego, Calif.) software.

### Example 4

### Competitive Binding Assays With Aβ Oligomers And Aβ Monomer

A competitive binding assay with Aβ oligomers and Aβ monomer demonstrated a preference for Aβ oligomers binding using the 19.3 antibody. Aβ oligomers plates were prepared as above in Example 3, through the Casein buffer blocking step. Aβ40 monomer-coated plates were prepared in the same way, using 100 pmol/well. The 19.3 antibody, from Example 2, was applied at 4 nM (EC50 for Aβ oligomers as determined in Example 3 above) to each well in the Casein blocking buffer matrix and allowed to interact with Aβ oligomers or Aβ40 for 30 minutes at room temperature with shaking. A 12-point, three-fold concentration curve starting at 10 μM, for either Aβ oligomers or Aβ40, was applied to the antibody containing wells. For plates coated with Aβ oligomers, Aβ40 was added to the wells; for Aβ40 plates, Aβ oligomers were added to the wells. The plates were incubated for one and half hours at room temperature. Both detection of residual antibody binding and the EC50 calculations were determined in Example 3 above.

### Example 5

### Sandwich ELISA On Envision Platform

A. Aβ oligomers Assay: Sandwich ELISAs were applied to the complete Aβ oligomers preparation or human CSF. The 19.3 Aβ oligomer-preferring antibody was coated at 0.5 μg per well in sodium bicarbonate buffer (ThermoFisher #28382, Waltham, Mass.) overnight at 4° C. Next day, the wells were washed with phosphate-buffered saline with 0.05% Tween 20 (PBS-T) and blocked overnight at 4° C. with 200 μL/well casein buffer in PBS (ThermoFisher #37528, Waltham, Mass.), with 0.1% Tween added. Aβ oligomer standards or SEC fractions were diluted in casein buffer and added at 100 μL/well. Dilutions providing signal in the linear range of the standard curve were used for calculations. Next day, plate was washed five times with PBS-T and Biotin-82E1 (IBL, No.10326, Toronto, Ontario, Canada) was added at 100 μl/well in casein buffer for one hour at room temperature. The plates were washed again with PBS-T and Neutravidin-AP (ThermoFisher #31002, Waltham, Mass.) was added for 30 minutes at room temperature. Finally, after additional PBS-T washes, Tropix® CDP®-Star chemiluminescent substrate (Life Technologies™, Carlsbad, Calif.) was added for 30 minutes. Luminescence was quantified on an EnVision® (PerkinElmer, Waltham, Mass.) plate reader.

B. Aβ monomer assay: Aβ40 (American Peptide Co, Sunnyvale, Calif.) was dissolved in 1,1,1,3,3,3 hexafluoro-2-propanol (HFIP, Sigma-Aldrich, St. Louis, Mo.). The HFIP was removed by evaporation, and the dried peptide film was then re-dissolved in dimethyl sulfoxide (DMSO, Sigma Aldrich, St. Louis, Mo.). Standard method for carrying out an ELISA and/or biotinylation of reagents can be found in Antibodies: a Laboratory Manual, Cold Spring Harbor Laboratory Press, Cold Spring Harbor, N.Y., Harlow E, Lane D (1988)). Methods for detection of Aβ monomers using a sandwich ELISA protocol have been previously reported (Sankaranayaranan et al., J. Pharmacol. Exp. Ther., 328:131-140) using commercially available antibodies, such as 6E10, 12F4 and G210 (Covance, Princeton, N.J.).

### Example 6

### Human CSF Samples

CSF samples from clinically-confirmed AD, young control, or age-matched control patients were purchased from BioReclamation (Hicksville, N.Y.) or Precision Med (Solana Beach, Calif.). The cognitive diagnosis was made using the commonly accepted Mini-Mental State Exam (MMSE). The nature of the samples was confirmed by respective measure of Aβ40 and Aβ42 monomer by ELISA (Example 5B), which has been reported either unchanged, or significantly reduced in AD CSF.

### Example 7

### Pharmacokinetic Analysis of 19.3 In Non-Human Primates

To confirm the presence of 19.3 in primate CSF, a study was conducted for the anti-Aβ oligomer antibody 19.3 in a cohort of cisterna magna ported rhesus monkeys. Six animals (three male/three female) were dosed with a single intravenous bolus of antibody 19.3 (20 mg/kg). CSF samples were collected from the cisterna magna port at various time points and the concentration of antibody 19.3 in the CSF was determined with an anti-human IgG ELISA assay. Applicants found that antibody 19.3 was able to cross into the primate CSF, where it increased in concentration during the first 24 hours and peaked at about 100 ng/mL. This concentration guided the level of anti Aβ oligomer antibody 19.3 spiked into the human CSF for development of the target engagement assay.

### Example 8

Aβ oligomers sandwich ELISA paramagnetic micro-particle based immumoassay The Aβ oligomer sandwich ELISA was carried out using a paramagnetic microparticle-based immumoassay platform (Erenna® immunoassay system, Singulex®, Almeda, Calif.) to determine oligomer levels in human samples or Aβ oligomer standards. Micro-particles (MPs) for capture were prepared by binding 12.5 μg of the capture reagent, Aβ oligomer antibody 19.3, per mg of MPs (see method below). The 19.3 bound MPs were diluted to 100 μg/mL in assay buffer (Tris buffer with 1% Triton X-100, d-desthiobiotin, 0.1% bovine serum albumin) and added at 100 uL to 100 uL of CSF sample or standards (diluted in Tris buffer and 3% bovine serum albumin), followed by incubation for two hours at 25° C. The MPs were retained via a magnetic bed, and unbound material was removed in a single wash step using assay diluent using the THydroflex plate washer (Tecan, Männedorf, Switzerland). The alexa-fluorescent-labeled detection antibody, 82E1 (prepared as example below), was diluted to a final concentration of 500 pg/mL and filtered through a 0.2 μm filter (Pall 4187, Fort Washington, N.Y.). The antibody was added to 20 μL/well of individual sample particles. The ELISA plates were incubated for one hour at 25° C., while shaking in a Jitterbug (Boekel, Feasterville, Pa.). The wells were washed four times with assay buffer to remove any unbound detection reagent. MP/19.3/Aβ oligomer/82E1 complexes were transfered to a new plate, buffer was aspirated off and 10 μL/well of elution buffer was added, followed by a 5 minute incubation at 25° C., while shaking in a Jitterbug at speed 5. Eluted, fluor-labeled detecting antibody 82E1 was transferred to a 384 plate containing 10 μL/well neutralization buffer and read on a paramagnetic micro-particle detector (Erenna®, Singulex®, Alameda, Calif.) at 60 seconds per well read time.

### A. Capture Antibody Labeling

1. Binding of Aβ oligomer antibody (19.3) to Dynabeads (MP beads): Remove supernatent from 50 μl Dynabeads using magnet. Resuspend Dynabeads in 200 μl of an antibody binding and washing buffer, such as RIPA buffer [#9806, Cell Signaling Technologies, Beverly, Mass.], containing 5 μg of 19.3. Incubate for 10 minutes with rotation at room temperature. Remove supernatent from 19.3/MP bead complex with a magnet. Wash the complex with 200 μl of the binding and washing buffer.

2. Coupling of Aβ oligomer antibody (19.3) to Dynabeads (MP beads): Just prior to use, make 5 mM BS3 solution (Bis(sulfosuccinimidyl)suberate, Cat. # 21580 Thermo Fisher Scientific Inc., Waltham, Mass.) in a conjugation buffer (20 mM Sodium Phosphate, 0.15 M NaCl (pH 7-9)); 250 μl of this solution is required per sample (5 μg 19.3/50 μl MP bead complex). Wash the 19.3 coupled MP beads (19.3/MP beads) twice in 200 μL of the conjugation buffer. Place on a magnet and discard supernatant. Resuspend the 19.3/MP beads in 250 μl 5 mM BS3. Incubate at room temperature for 30 minutes with tilting/rotation. Quench the cross-linking reaction by adding 12.5 μl of a quenching buffer (1M Tris HCl (pH 7.5)) and incubate at room temperature for 15 minutes with tilting/rotation. Wash the cross-linked MP beads three times with 200 μl PBST. Dilute the MP beads to 100 μg/mL in Assay buffer for use as in above assay protocol.

### B. Detection Antibody Labeling

Coupling Alexa Fluor 546 to 82E1: 82E1 was coupled to a fluorescent tag comparable to Alexa Fluor 546 (Invitrogen, Carlsbad, Calif.), according to the manufacturer's protocol. Briefly, 82E1 was diluted to 1 mg/mL and one-tenth volume of 1M sodium bicarbonate buffer was added. This solution of 82E 1 (100 μL) was added to the vial of Alexa Fluor 546 dye, and the vial was capped, gently inverted to dissolve the dye and stirred at room temperature for 1 hour. Spin the columns to separate any unlabeled fluorescent tag from the detection antibody, while loading the Component C (BioGel® P-30, BioRad, Hercules, Calif.) fine size exclusion purification resin onto the column. After the gel buffer drains away, 100 μL 19.3/MP beads and dye reaction volume was added onto the center of the resin at the top of the spin column and absorbed into the gel bed. To the column was slowly added at room temperature 100 μL of an elution buffer (0.01 M potassium phosphate, 0.15 M NaCl, pH 7.2, with 0.2 mM sodium azide). Additional elution buffer was added and as the column ran, the column was illuminated to visualize the front of the dyed/tagged antibody. The first column dye line is the labeled antibody. Free dye remains in the column bed, discard the spin column.

Human CSF samples were obtained from Bioreclamation (Hicksville, N.Y.) and after thawing were kept on ice. The CSF samples were treated with 0.05% Tween-20, (2.5% Tween-20 stock diluted 1:50 into CSF) prior to sampling. Samples or Aβ oligomer standards were diluted into Tris buffered saline (TBS) with 3% bovine serum albumin (BSA). MPs coupled with 19.3 were diluted to a final concentration of 100 μg/mL in an assay buffer containing TBS/0.1% BSA/1.0% Triton X-100. To each well of a 96-well plate was added 100 μL sample/standard and 100 μL 19.3-coated MPs. Samples were incubated with MPs for 60 minutes at room temperature (RT) and washed once using magnetic separation following the addition of TBS/0.1% BSA/1.0% Triton X-100 MP re-suspension buffer. The fluorescently-labeled detection antibody, 82E1, was added at 20 μL per well and incubated for 30 minutes at room temperature, followed by four washes with the MP re-suspension buffer using magnetic separation. The fluorescent tag was eluted off the detection antibody 82E1 and incubated five minutes at room temperature. The eluate was transferred to a microplate containing a neutralization buffer and transferred to a detection device capable of reading the magnetic micro-particles (MPs), such as the Erenna® instrument (Singulex®, Almeda, Calif.) at 60 seconds per well read time.

### Example 9

### Aβ Oligomer Complex Sandwich ELISA Target Engagement (TE) Assay

An Aβ oligomer complex sandwich ELISA can be carried out for use as a target engagement assay to detect antibody/Aβ oligomer complexes formed in vitro or in vivo, for use with a therapeutic antibody to show target engagement or to demonstrate efficacy of a therapeutic antibody to reduce 19.3/Aβ oligomer complexes. Either anti-human IgG2 or anti-human kappa (both from Southern Biotech, Birmingham, Ala.) were coated at 0.5 μg per well in a sodium bicarbonate buffer overnight at 4° C. (BupH Carbonate-Bicarbonate Buffer pack, #28382, Thermo Fisher Scientific Inc, Waltham Mass. Next day, the wells were washed with a phosphate-buffered saline with 0.05% Tween 20 (PBS-T; Sigma-Aldrich, St. Louis, Mo.) and blocked overnight at 4° Celcius with 200 μL/well casein buffer in PBS with 0.1% Tween added. The 19.3 antibody was spiked into a Casein buffer (Thermo Fisher Scientific Inc, Waltham Mass.) or human CSF in microcentrifuge tubes (Axygen, Inc., Union City, Calif., MCT-175-L-C) at 0.100 μg/mL. The Aβ oligomers were spiked at varying concentrations to give a standard curve, keeping the 19.3 levels constant. The samples were agitated at 4° C. for one hour to enable formation of the antibody (19.3)/Aβ oligomer complexes. 100 μl sample/well was applied to either an anti-human IgG2 or an anti-human kappa-coated plate (n=3) and incubated overnight at 4° C. on a plate shaker. Next day, the plates were washed five times with PBS-T and Biotin-82E1 (IBL, Minneapolis, Minn., No. 10326) was added at 100 μl/well, diluted 1:5000 in Casein blocking buffer (Sigma-Aldrich Corp., St. Louis, Mo.), 0.1% Tween 20 for one hour at room temperature. The plates were washed again with PBS-T, and Neutravidin-AP (ThermoFisher, Waltham, Mass., #31002) was diluted 1:20,000 in Casein buffer, then added for 30 minutes at room temperature. Additional PBS-T washes were followed with Tropix CDP star luminescence substrate (Applied Biosystems, Foster City, Calif., T2214) applied for 30 minutes. Luminescence was quantified on an EnVision plate reader (PerkinElmer, Waltham, Mass.).

What is claimed:

1. A method for determining the level of a neuronally derived protein of interest (NDPOI) in a biological sample obtained from a patient, comprising:
(a) obtaining a biological sample having a NDPOI from a mammal;
(b) contacting said biological sample with a capture antibody/paramagnetic micro-particle bead (antibody/MP bead) under conditions sufficient to form a NDPOI/capture antibody/MP bead complex;
(c) contacting the NDPOI/capture antibody/MP bead complex of step (b) with a fluorescently labeled detection antibody under conditions sufficient to form POI/capture antibody/MP bead/detection antibody complex; and
(d) detecting the fluorescent signal generated from said complex of step (c); wherein the fluorescent signal of step (d) represents the amount of the NDPOI.

2. A method of claim 1 wherein the NDPOI is an Aβ oligomer.

3. A method of claim 1 wherein the mammal is a human.

4. A method of claim 1 wherein the capture antibody is an anti-Aβ oligomer antibody selected from the group consisting of 19.3, 7305, 82E1, and W02.

5. A method of claim 1 wherein the detection antibody is an anti-Aβ oligomer antibody selected from the group consisting of 82E1, 7305, and 6E10.

6. A method of claim 1 wherein the capture antibody is 19.3 and the detection antibody is 82E1.

7. A method of claim for identifying a patient having Alzheimer's disease by determining the level of a neuronally derived protein of interest (NDPOI) in a biological sample obtained from a patient, wherein the NDPOI is an Aβ oligomer, and wherein patients having Aβ oligomer levels ranging from 0.5 pg/mL to 11 pg/mL are determined to have Alzheimer's disease.

8. A method for determining the therapeutic efficacy of a therapeutic to treat Alzheimer's disease comprising:
(a) obtaining a biological sample having a neuronally derived protein of interest (NDPOI) from a patient;
(b) contacting said biological sample with a capture antibody/paramagnetic micro-particle bead (antibody/MP bead) under conditions sufficient to form a NDPOI/capture antibody/MP bead complex;
(c) contacting the NDPOI/capture antibody/MP bead complex of step (b) with a fluorescently labeled detection antibody under conditions to form NDPOI/capture antibody/MP bead/detection antibody complex; and
(d) detecting the fluorescent signal generated from said complex of step (c) and where the fluorescent signal represents the amount of the NDPOI;
(e) administering a test therapeutic to said patient in need thereof;
(f) obtaining a second biological sample having a NDPOI from said patient;
(g) repeating steps (b) through (d) with the second biological sample from said patient; and
(h) comparing the fluorescent signal detected from the second biological sample to said signal from the first biological sample;
wherein a decrease in the fluorescent signal detected represents an effective therapeutic.

9. The method of claim 8 wherein the NDPOI is an Aβ oligomer.

10. The method of claim 8 wherein the capture antibody is 19.3 and the detection antibody is 82E1.

11. A method for determining the target engagement of a therapeutic antibody bound to a neuronally derived protein of interest (NDPOI) comprising:
(a) administering a therapeutic antibody to a mammal;
(b) obtaining a biological sample having a NDPOI from said mammal;
(c) contacting said biological sample with a capture antibody/paramagnetic micro-particle bead (antibody/MP bead) under conditions sufficient to form a NDPOI/capture antibody/MP bead complex;
(d) contacting the NDPOI/capture antibody/MP bead complex of step (b) with a fluorescently labeled detection antibody under conditions to form NDPOI/capture antibody/MP bead/detection antibody complex; and
(e) detecting the fluorescent signal generated from said complex of step (c) and wherein the fluorescent signal represents the target engagement of the NDPOI/therapeutic antibody.

12. A method of claim 11 wherein the NDPOI is an Aβ oligomer.

13. The method of claim 11 wherein the capture antibody is 19.3 and the detection antibody is 82E1.
